(19) United States
(12) Patent Application Publication
(10) Pub. No.: US 2019/0295727 A1
(43) Pub. Date: Sep. 26, 2019

(54) System and Method for Assessing Heart Health and Communicating the Assessment to a Patient

(71) Applicant: American Heart Association, Inc., Dallas, TX (US)

(72) Inventors: Vanessa Xanthakis, Melrose, MA (US); Danielle Enserro, East Amherst, NY (US); Vasan Ramachandran, Berlin, MA (US); Robyn McClelland, Seattle, WA (US); Bridget Kruszka, Pittsburgh, PA (US); Ralph Sacco, Miami Beach, FL (US); Chuanhui Dong, Miami, FL (US); Adolfo Correa, Jackson, MS (US); Eduardo Sanchez, Dallas, TX (US)

(21) Appl. No.: 16/360,747

(22) Filed: Mar. 21, 2019

Related U.S. Application Data
(60) Provisional application No. 62/647,020, filed on Mar. 23, 2018.

(51) Int. Cl. G16H 50/30; G16H 80/00; A61B 5/00

(52) U.S. Cl. CPC G16H 50/30; G16H 50/20; A61B 5/7275; G16H 80/00

(57) ABSTRACT

A method and system for assessing a patient's heart health and communication that assessment to the patient in an understandable format. Various personal data and information regarding personal risk factors are used to calculate a CVH score. The CVH score, patient's actual age, and various predetermined functional parameters specific to the patient's race and sex are used to calculate a vascular age of the patient. The vascular age is this provided to the patient and may be older than the patient's actual age (indicating poor cardiovascular health), the same as the patient's actual age (indicating average cardiovascular health), or younger than the patient's actual age (indicating ideal cardiovascular health). Optionally health information may also be provided based on the personal risk factors, CVH score, vascular age, or comparisons to previous values or predetermined thresholds.

### CROSS-REFERENCE TO RELATED APPLICATION

This application claims the benefit of U.S. Provisional Application Ser. No. 62/647,020 filed on Mar. 23, 2018.

### BACKGROUND OF THE INVENTION

### 1. Field of the Invention

This invention relates to a system and method for assessing a patient's heart health and communicating such assessment in an easily understandable format and optionally with heart health related information to aid the patient in improving his or her heart health.

### 2. Description of Related Art

A problem frequently encountered by healthcare providers is how to adequately convey to patients in an understandable form the status of the patients' health. Various tests may be performed that provide numerical results, such as blood pressure measurements and total cholesterol, but the significance of these numerical results may not be easily understood by the patient and therefore may not adequately convey to the patient as assessment of the patient's health. There are several known methods of assessing a patient's health that involve determining a health score that are intended to be more easily understood than raw test measurement data. For example, U.S. Pat. No. 9,378,335 discloses a system and method for determining a health score based on various health related information, comparing the health score to a threshold, and taking action based on the comparison, such as providing the user with a list of food items that if avoided would improve the health score. As another example, the American Heart Association's My Life Check determines a heart health score, on a scale up 10, based on various health related information. Although the My Life Check score is easy to calculate, patients do not necessarily understand the meaning of the score and healthcare providers and clinicians find it difficult to convey the meaning of the score to the lay person or patient, since the a numerical score is not inherently meaningful in conveying CVD. Most patients are able to understand that having a low My Life Check score translates to higher chance of developing CVD, but it is not easy for a patient to understand how much he/she needs to improve their My Life Check score to avoid developing CVD, and how such knowledge can motivate patients towards improved cardiovascular health. Additionally, the clinical significance of a My Life Check score in the intermediate range is not readily obvious either in terms of motivating lifestyle changes or the likelihood of developing CVD over the short-term.

Other known health assessment tools involve graphical representations of health, rather than numerical representations. For example, U.S. Pat. No. 9,101,261 discloses using health information regarding a patient to generate an avatar image designed to illustrate the patient's health status using different facial expressions and characteristics on the generated avatar. As another example, U.S. Pat. No. 9,241,677 discloses variations on smiley faces, stop light graphics, and graphical hearts of varying sizes to communicate a patient's health status.

Although these prior methods of communicating health assessments are beneficial, there is still a need for other alternatives and more easily understandable assessment methods. There is also a need to more accurately assess a patient's heart health based on race and sex specific parameters.

### SUMMARY OF THE INVENTION

A method and system according to a preferred embodiment of the invention allows a user to input certain personal information regarding a patient, including race, sex, and age, and information regarding one or more personal risk factors for the patient, in order to obtain an assessment of the patient's heart health in an easily understandable format. Preferably, the personal risk factors include one or more from the following categories: physical risk factors, medical risk factors, and lifestyle risk factors. According to one preferred embodiment, the user's inputs are compared to ideal values to calculate a cardiovascular health (CVH) score for the patient, which may optionally be displayed or provided to the user or patient. According to another preferred embodiment, the system and method retrieve certain data from a pre-populated database to use in calculations, along with the patient's calculated CVH score, to assess the patient's overall heart health and to display or provide that assessment to the user or patient in an easily understandable format.

According to one preferred embodiment, an easily understandable format for the patient's heart health is a number that represents the patient's cardiovascular age, which may be around the same, younger, or older than the patient's actual age. A younger cardiovascular age indicates good or even ideal cardiovascular health. An older cardiovascular age indicates poor cardiovascular health. A cardiovascular age around the same as the patient's actual age indicates average cardiovascular health for the patient's age. According to another preferred embodiment, the easily understandable format is a score on a scale of 1 to 10 that corresponds to the American Heart Association's My Life Check® scale.

According to another preferred embodiment, a system and method of the invention provides the user with health related information, such as educational support materials, corresponding to physical, medical, and/or lifestyle risk factors. According to another preferred embodiment, such health related information is provided when one or more of the user's inputs indicate that the patient has a moderate to high risk in a given category or otherwise indicates that the patient's information is less than ideal or above or below of predetermined threshold. According to another preferred embodiment, a system and method of the invention provides the user with generalized health information.

According to another preferred embodiment of the invention, the patient's personal data, personal risk factor data, previous CVH score and/or previous vascular age are stored for future use. Comparisons to previous data or scores or vascular age may be used to provide information to the user regarding changes in the patient's heart health status.

### BRIEF DESCRIPTION OF THE DRAWINGS

The method of the invention is further described and explained in relation to the following drawings wherein:

FIG. 1 is a flow chart showing process stages for a preferred embodiment of the heart health assessment system and method of the invention;

FIG. 2 is a flow chart showing preferred process steps for one preferred stage of the embodiment according to FIG. 1;

FIG. 3 is a flow chart showing preferred process steps for certain stages of the embodiment according to FIG. 1;

FIG. 4 is a flow chart showing preferred process steps for certain sub-stages of the embodiment according to FIGS. 2-3;

FIG. 5 is a flow chart showing preferred process steps for certain sub-stages of the embodiment according to FIGS. 2-3;

FIG. 6 is a flow chart showing preferred process steps for certain sub-stages of the embodiment according to FIG. 3;

FIG. 7 is a flow chart showing preferred process steps for certain sub-stages of the embodiment according to FIGS. 2-3;

FIG. 8 is a flow chart showing preferred process steps for certain sub-stages of the embodiment according to FIG. 3;

FIG. 9 is a flow chart showing preferred process steps for certain sub-stages of the embodiment according to FIG. 3;

FIG. 10 is a flow chart showing preferred process steps for certain sub-stages of the embodiment according to FIG. 1;

FIGS. 11A-11C are continuing flow charts showing preferred process steps for a certain sub-stage of the embodiment according to FIG. 1;

FIG. 12 is a flow chart showing process stages for another preferred embodiment of the heart health assessment system and method of the invention;

FIG. 13 is a flow chart showing additional process stages for the embodiment of FIG. 12;

FIG. 14 is a table showing sample exclusion from the cohorts.

### DESCRIPTION OF THE PREFERRED EMBODIMENTS

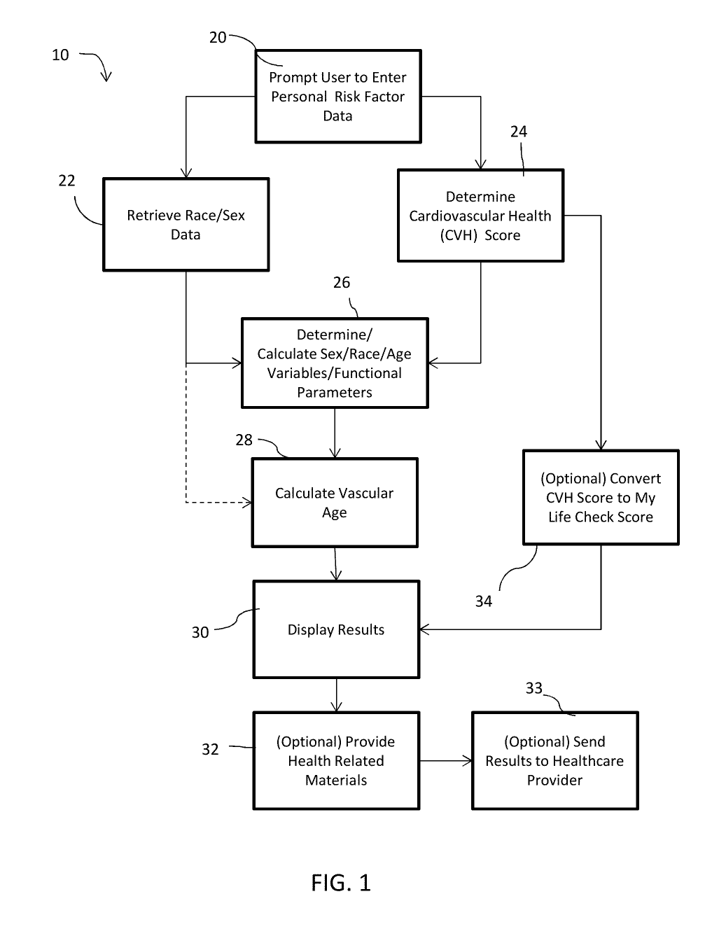

A preferred embodiment of a method and system 10 for assessing and communicating a patient's cardiovascular health is shown in FIGS. 1-11C. The method and system 10 includes several processing stages, including entry of the patient's personal data 20, retrieval of race/sex data from database/table 22, determining cardiovascular health (CVH) score 24, determining or calculating sex/race/age variables or functional parameters 26, calculating vascular age 28, optionally converting CVH score to a My Life Check Score 34, displaying the results 30, optionally providing health related materials 32, and optionally sending results to a healthcare provider 33. Typically, the user will be the patient, but a third party (such as a family member or caretaker) or healthcare provider may also be the user to enter data on behalf of a patient.

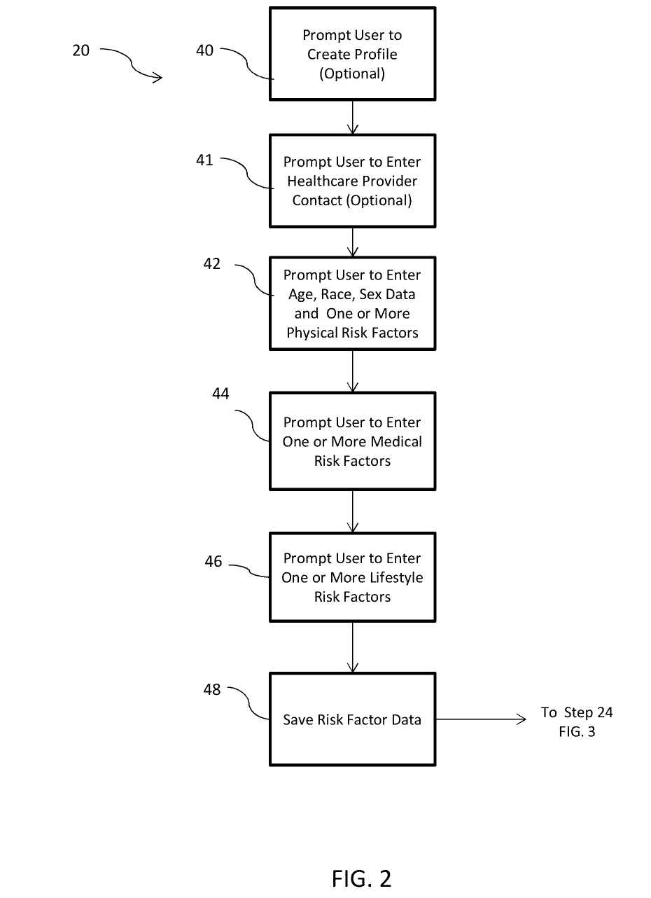

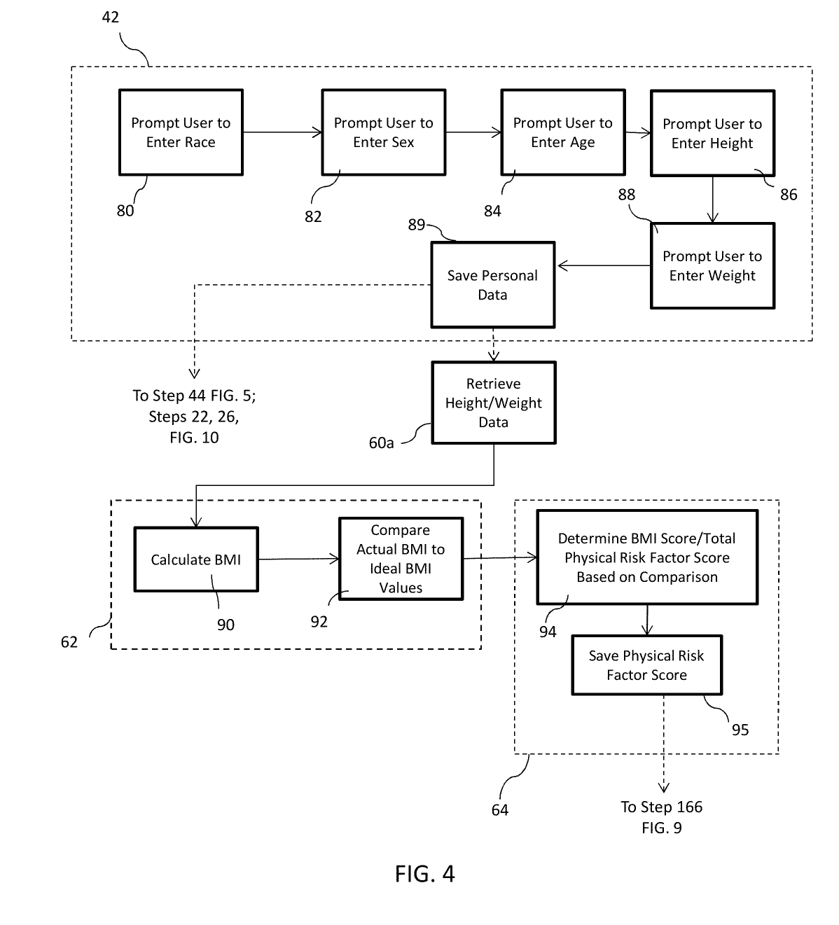

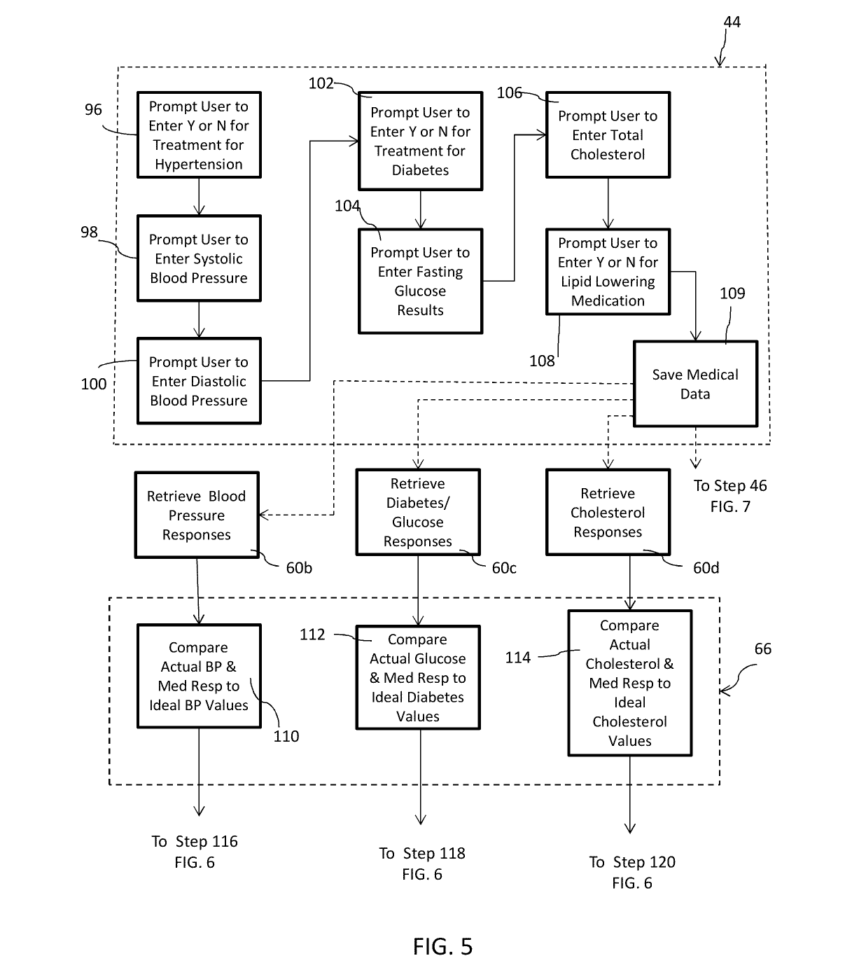

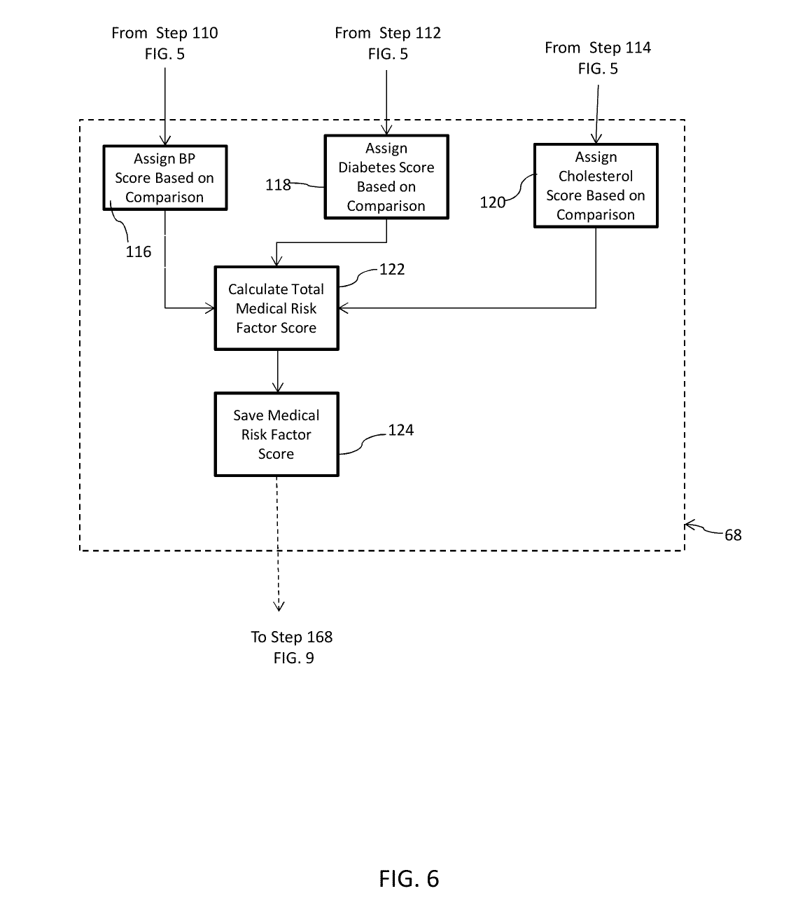

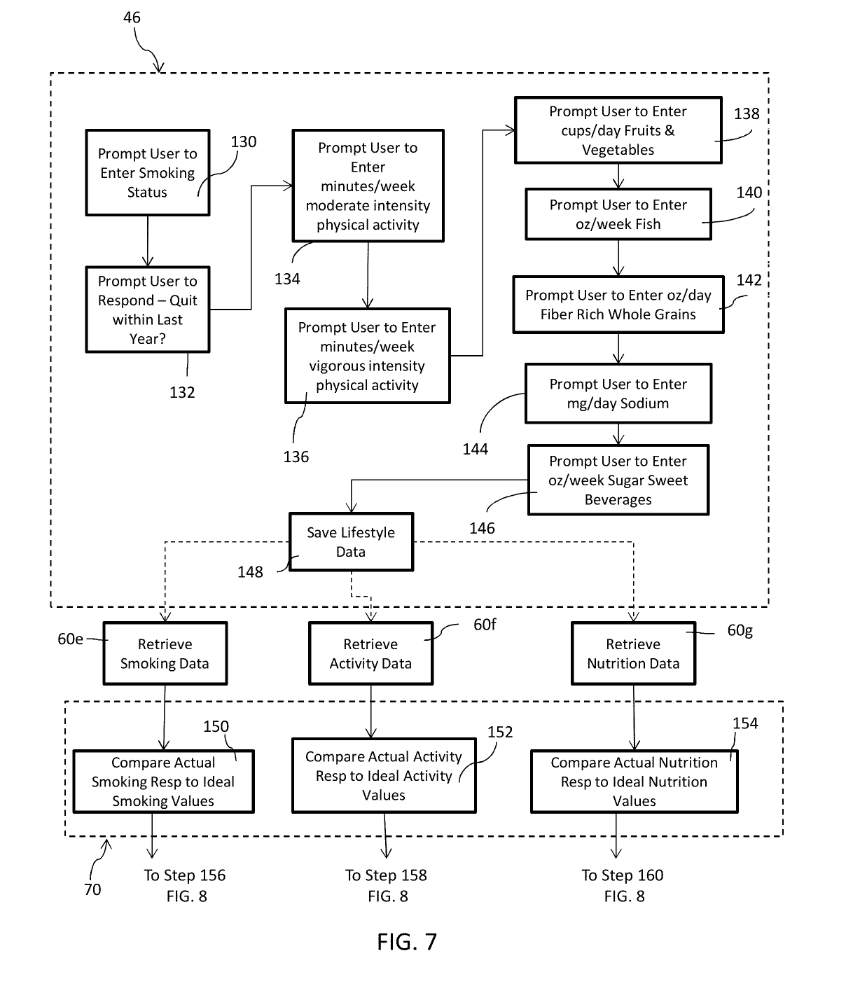

As shown in FIG. 2, a personal data entry stage 20 preferably comprises optionally prompting the user to create a profile 40, optionally prompting the user to enter healthcare provider contact information 41 so that results of the calculations may be optionally shared with the healthcare provider at step 33, prompting the user to enter age, race, sex information and data regarding one or more physical risk factors 42 (described in more detail in FIG. 4), prompting the user to enter data regarding one or more medical risk factors 44 (described in more detail in FIG. 5), prompting the user to enter data regarding one or more lifestyle risk factors 46 (described in more detail in FIG. 7). Optionally, but preferably, a user will create a profile at step 40 by entering a user name and password. A user name should be unique to each user, so system 10 may compare and entered user name to existing user names to make sure there are no duplicates. If a user attempts to use a duplicate user name, he/she will be prompted to enter a different user name until a unique user name is entered. Criteria for user name and password may be pre-set to require a certain number of characters, numbers, or special characters, or to prohibit the use of certain characters, as is standard in user profile creation. A user may also be periodically required to change his/her password, if desired.

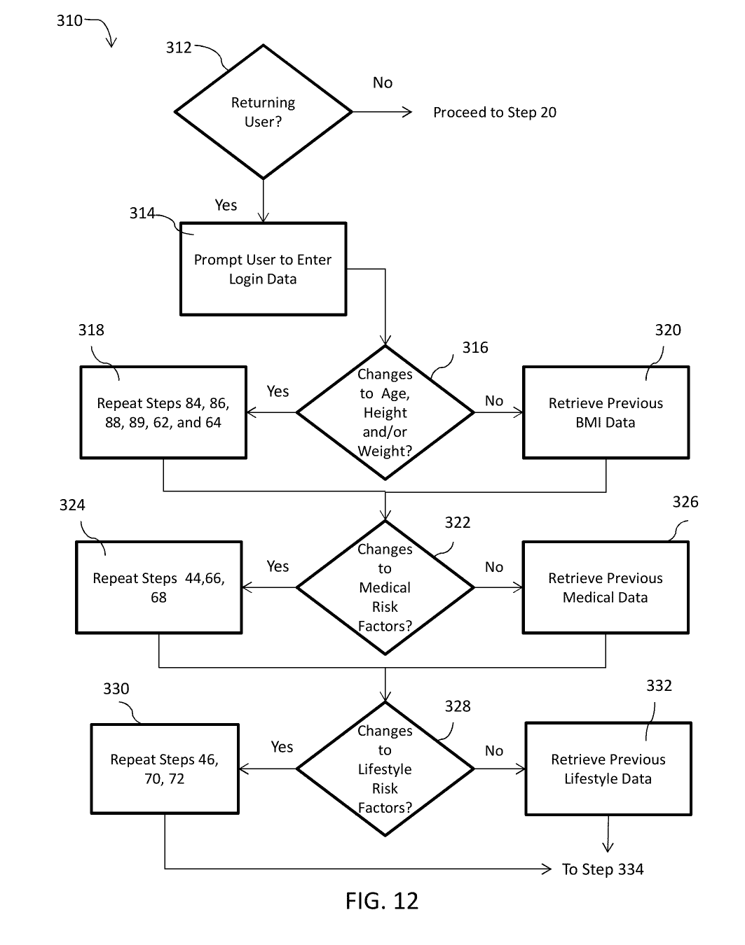

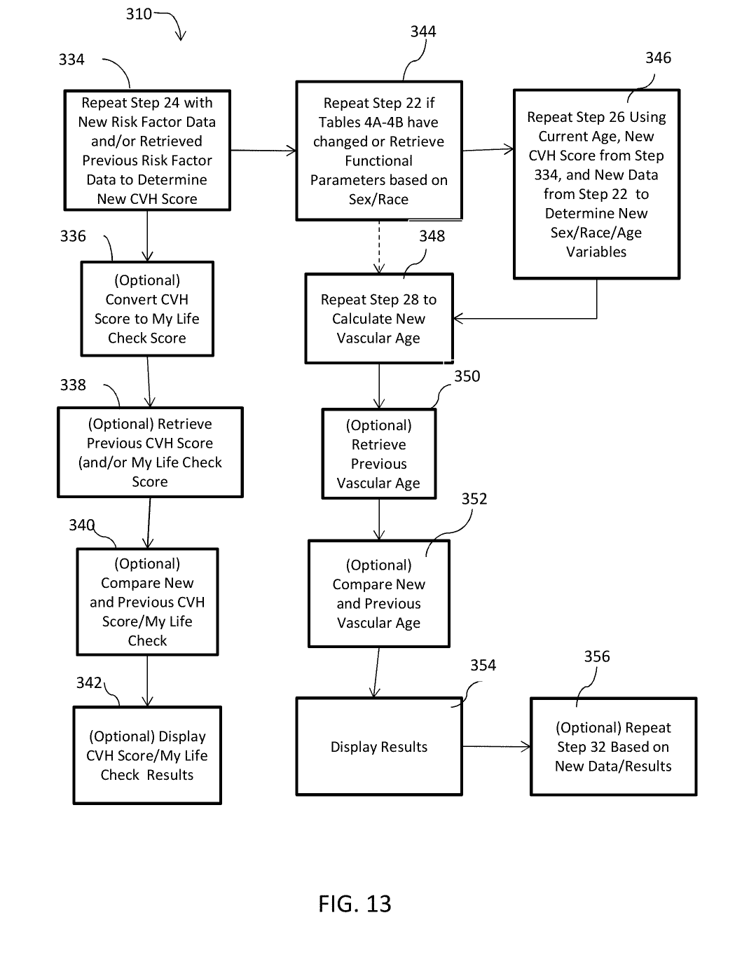

The data entered is preferably stored at step 48 so that it may be retrieved for use in making calculations and completing data lookup/retrieval tasks as part of steps 22, 24, 26, 28, and optional step 32. All saved data or data storage in connection with system 10 described herein may be short term or temporary storage, for use only during the current session as need to make calculations and complete data lookup tasks to provide the user with the patient's vascular age results. If the patient has created a profile at step 40, then such storage may be long term, to allow the patient to later retrieve the vascular age results and to repeat the calculations according to another preferred embodiment of a method and system 310 according to the invention, as shown in FIGS. 12-13.

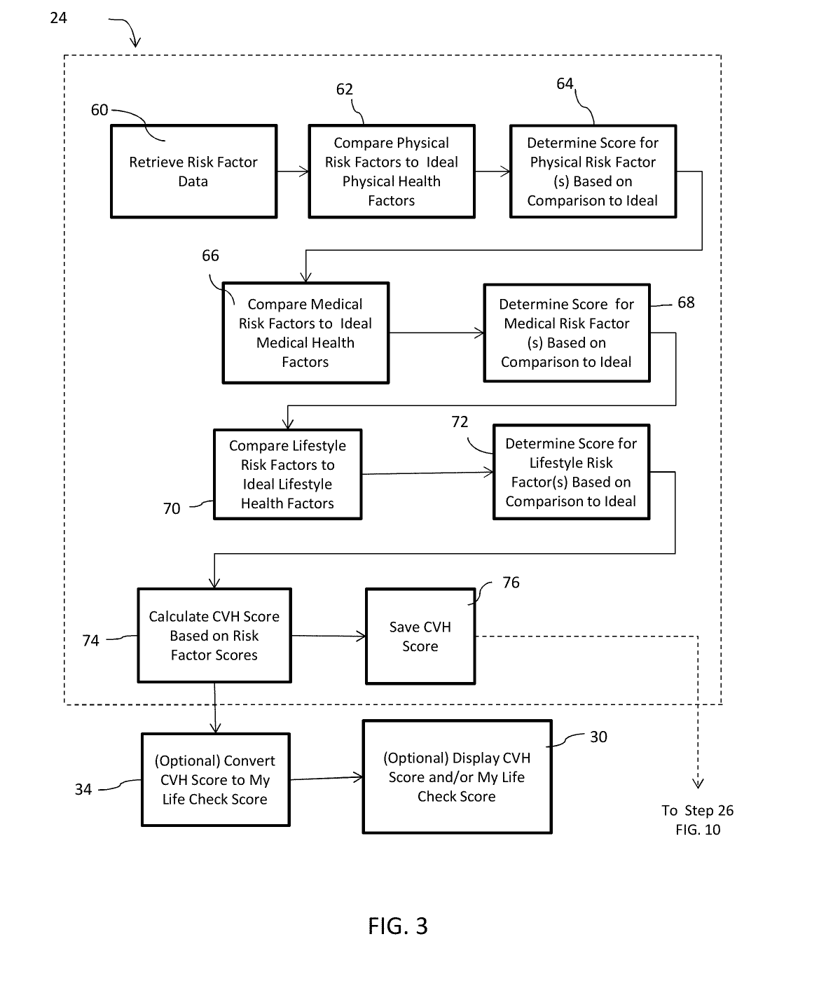

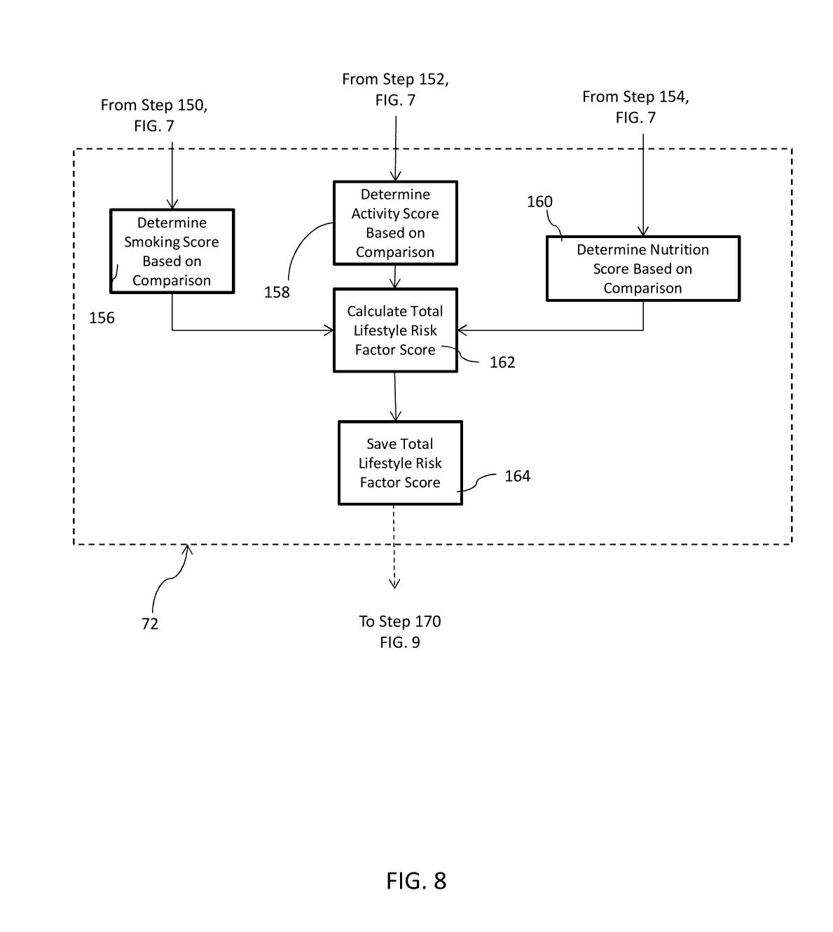

As shown in FIG. 3, a CVH score determination stage 24 preferably comprises retrieving risk factor data at step 60 and comparing the retrieved data to ideal data steps 62 (described in more detail in FIG. 4), 66 (described in more detail in FIG. 5), and 70 (described in more detail in FIG. 7) in order to determine scores at steps 64 (described in more detail in FIG. 4), 68 (described in more detail in FIG. 6), and 72 (described in more detail in FIG. 8). The scores determined at steps 64, 68, and 72 are added together to calculate a CVH score at step 74. Optionally, the CVH score may be converted to a My Life Check score at step 34 using a simple mathematical conversion from a 0-14 scale to a 0-10 scale (e.g. a CVH score of 14 equals a My Life Check Score of 10, a CVH score of 7 equals a My Life Check score of 5) and the CVH score and/or My Life Check Score may be displayed to the user at step 30. The CVH score is also saved or stored at step 76 for later use.

Referring to FIG. 4, a user is preferably prompted to enter basic physical information regarding the patient at step 42. Such information includes personal data regarding the patient's race 80, sex 82, and age 84, and physical risk factors relevant to cardiovascular health, such as height 86 and weight 88. Other data that may be relevant to cardiovascular health, such as specific ethnic origin information, may also be included in step 42. This data is preferably saved at step 89 for later use and retrieved at step 60a. At step 62, the physical risk factor data (such as height and weight or a calculated BMI) is compared to ideal data. Most preferably, step 62 comprises calculating a BMI based on the patient's height and weight data retrieved at step 60a. The patient's actual BMI is compared to ideal BMI at step 92 and a score is determined at step 94 based on the comparison. The preferred criteria for determining the BMI/total physical risk factor score is discussed further below. The physical risk factor score is saved at step 95 for later use in calculating the CVH score. BMI is the preferred physical risk factor for use in calculating CVH score, but other physical risk factors may also be used if desired.

Referring to FIGS. 5 and 6, a user is preferably prompted to enter basic medical information regarding the patient at step 44. Such information includes risk factors for cardiovascular health, such as whether the patient is taking medication to control blood pressure 96, the patient's systolic blood pressure (SBP) 98, the patient's diastolic blood pressure (DBP) 100, whether the patient is taking medication to control diabetes 102, the patient's fasting glucose results 104, the patient's total cholesterol 106, and whether the patient is taking medication to control cholesterol or lower lipids 108. This data is preferably saved at step 109 for later use and retrieved at steps 60b-60d. These particular medical risk factor categories are preferred, but other medical risk factors may also be used if desired. For example, instead of total cholesterol, separate amounts for LDL and HDL may be used. Additionally, triglycerides may be an added medical risk factor. The patient's blood pressure data/response is compared to ideal blood pressure data at step 110 and a score is determined at step 116 based on the comparison. The patient's diabetes/glucose data/response is compared to ideal glucose data at step 112 and a score is determined at step 118 based on the comparison. The patient's cholesterol data/response is compared to ideal cholesterol data at step 114 and a score is determined at step 120 based on the comparison. The preferred criteria for determining the blood pressure, cholesterol, and diabetes/glucose risk factor scores is discussed further below. The scores for each of these subcategories is added together at step 122 to calculate a total medical risk factor score, which is saved for later use at step 124.

Referring to FIGS. 7 and 8, a user is preferably prompted to enter basic lifestyle information regarding the patient at step 46. Such information includes risk factors for cardiovascular health, such as whether the patient is a current smoker 130 or quit within the last 12 months 132, the patient's minutes of moderate intensity physical activity per week 134, the patient's minutes of vigorous intensity physical activity per week 136, cups per day of fruits and vegetables eaten 138, ounces per week of fish eaten 140, ounces per day of fiber rich whole grains 142, mg per day sodium 144, and ounces per week of sugar sweet beverages 146. This data is preferably saved at step 148 for later use and retrieved at steps 60e-60g. These particular lifestyle risk factor categories are preferred, but other lifestyle risk factors may also be used if desired. For example, ounces of red meat eaten per week, grams of saturated fat per week, and similar subcategories may also be used or alternatively be used. Most preferably, the prompts for minutes of moderate intensity and vigorous intensity physical activity at steps 134, 136 provide examples of the types of activity that are considered moderate and those that are considered vigorous to aid the user in entering accurate information. For example, walking, vacuuming, and mopping may be examples of moderate intensity activity. Running, swimming laps, and riding a bicycle may be examples of vigorous intensity activity. Additionally or as an alternative, heart rate ranges for a man and for a woman and/or for different age ranges may be provided as examples indicating whether an activity is moderate or vigorous and the user may compare the patient's actual heart rate during any given activity to the ranges to determine whether the activity is moderate or vigorous.

The patient's smoking data/response is compared to ideal smoking data at step 150 and a score is determined at step 156 based on the comparison. The patient's activity data is compared to ideal activity data at step 152 and a score is determined at step 158 based on the comparison. The patient's nutritional/dietary data is compared to ideal dietary data at step 154 and a score is determined at step 160 based on the comparison. The preferred criteria for determining the smoking, activity, and dietary risk factor scores is discussed further below. The scores for each of these subcategories is added together at step 162 to calculate a total lifestyle risk factor score, which is saved for later use at step 164.

The physical risk factor score assigned at step 94, the medical risk factor scores determined at steps 116, 118, and 120, and the lifestyle risk factors scores determined at steps 156, 158, and 160 are preferably determined based on the data provided in Table 1 below. Most preferably, a CVH score is a scale of 0 to 14. Each risk factor sub-category (e.g. BMI risk factor, smoking risk factor, blood pressure risk factor), is scored a 0, 1, or a 2 based on the comparisons at steps 92, 110, 112, 114, 150, 152, and 154 to the corresponding data in Table 1. A score of zero for any sub-category indicates poor health in that subcategory; a score of 1 in any subcategory indicates intermediate health in that subcategory; and a score of 2 in any subcategory indicates ideal health in that subcategory.

TABLE 1
Scoring Criteria for CVH Subcategories

| | Poor Health | Intermediate Health | Ideal Health |
|---|---|---|---|
| Goal/Metric | (Score 0) | (Score 1) | (Score 2) |
| Physical Risk | ≥30 kg/m² | 25-<30 kg/m² | <25 kg/m² |
| Factor-Body | | | |
| mass index | | | |
| Lifestyle Risk | Current | Quit ≤ 12 months | Never or quit ≥ 12 |
| Factor-Smoking | smoker | | months |
| Lifestyle Risk | None | 1-149 min/week | >150 min/week |
| Factor-Physical | | moderate intensity | moderate intensity |
| activity* | | or | or |
| | | 1-74 min/week | >75 min/week |
| | | vigorous intensity | vigorous intensity |
| | | or | or |
| | | 1-149 min/week | >150 min/week |
| | | combined | combined |
| | | moderate/vigorous | moderate/vigorous |
| | | intensity | intensity |
| Lifestyle Risk | 0-1 | 2-3 components | 4-5 components |
| Factor-Healthy | components | | |
| diet score | | | |
| (see Table 2) | | | |
| Medical Risk | ≥240 mg/dL | 200-<240 mg/dL | <200 mg/dL and not |
| Factor-Total | | or | on lipid-lowering |
| cholesterol | | On lipid lowering | treatment |
| | | treatment/ | |
| | | medication to treat | |
| | | to goal | |
| Medical Risk | SBP ≥ 140 | SBP 120-<140 | SBP < 120 mmHg |
| Factor-Blood | mmHg or | mmHg or | and DBP < 80 |
| pressure | DBP ≥ 90 | DBP 80-<90 | mmHg and |
| | mmHg | mmHg or | not on hypertension |
| | | On hypertension | treatment |
| | | treatment/ | |
| | | medication to treat | |
| | | to goal | |
| Medical Risk | ≥126 mg/dL | 100-<126 mg/dL | <100 mg/dL and |
| Factor-Fasting | | or | not on diabetes |
| plasma glucose/ | | On diabetes | treatment |
| Diabetes | | treatment/ | |
| | | medication to treat | |
| | | to goal | |

Comparison step 154 preferably comprises several sub-steps 154a-154e where the patient's information entered at steps 138, 140, 142, 144, and 146 are compared to the data in Table 2 below. If the data entered at step 138 indicates the patient consumes 4.5 cups or more fruits and vegetable per day, then comparison step 154a passes, equating to a 1 component sub-score for that dietary element. If the data entered at step 138 indicates the patient consumes fewer than 4.5 cups of fruits and vegetables per day, then the comparison step 154a fails, equating to a zero sub-score for that dietary element. Similar pass/fail comparisons 154b-154e are made for each dietary element in table 2. At step 160 a nutrition score is preferably determined by adding up all of the component sub-scores from steps 154a-154e and comparing the total actual components to the ideal data in Table 1 to assign a nutrition/dietary score of 0, 1, or 2.

TABLE 2
Components for Health Diet Score

| Dietary Element | Amount for 1 Component |
|---|---|
| Fruits and vegetables (for step 138) | ≥4.5 cups per day |
| Fish (preferably oily fish) (for step | ≥two 3.5 oz. servings per week |
| 140) | |
| Fiber rich whole grains (for step | ≥1 oz. equivalent servings per day |
| 142) | |
| Sodium (for step 144) | <1500 mg/day |
| Sugar sweetened beverages (for step | ≤450 kcal per week |
| 146) | |

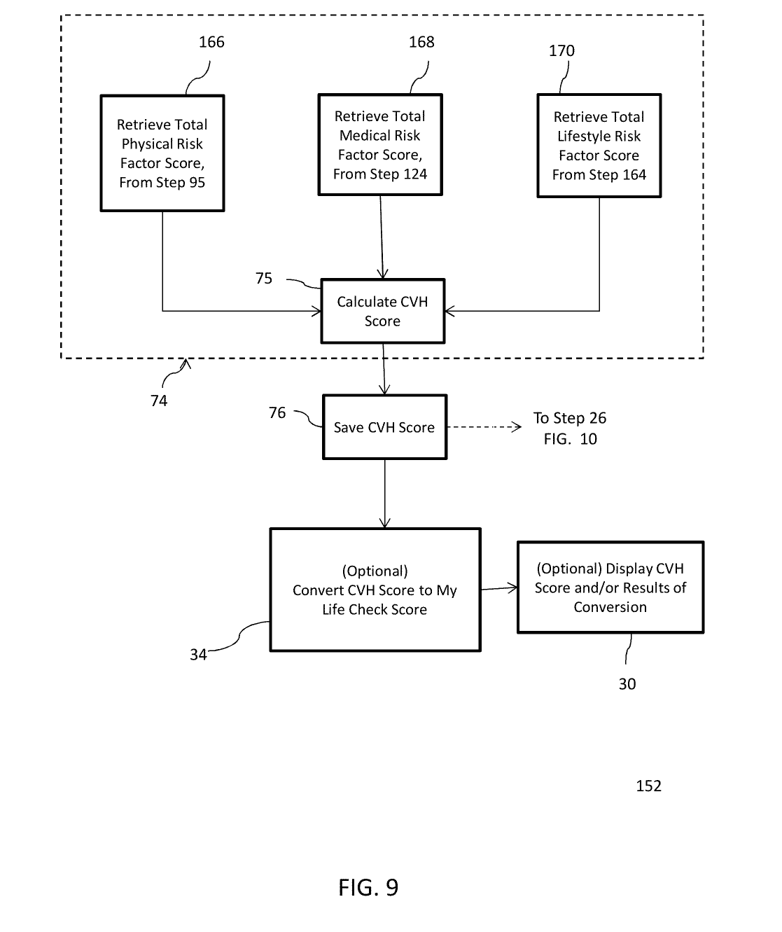

Referring to FIG. 9, the previously saved total physical risk factor, medical risk factor, and lifestyle risk factor scores are retrieved at steps 166, 168, and 170. These scores are added together at step 75 to calculate a CVH score, preferably on a scale of 0 to 14. Other scoring criteria or modifications to the criteria indicated in Table 1 may be used, if desired. The CVH score is saved at step 76.

Optionally, the CVH score may be converted to a My Life Check score, using a simple mathematical conversion from a 01-4 scale to a 0-10 scale, which estimates cardiovascular health on a scale of 1 (poor health) to 10 (ideal health) at step 34. Optionally, the CVH score and/or My Life Check Score may be displayed or otherwise provided, such as by email or printout, to the user/patient at step 30.

An example of the scoring for each category and CVH calculation based on hypothetical answers to the prompts in method and system 10 is provided below in Table 3.

TABLE 3
Example Scoring

| Goal/Metric | User's Response | Calculation | Score |
|---|---|---|---|
| Physical Risk Factor- | Height (step 86)-5′8″ | BMI (Step | 1 |
| Body mass index | Weight (step 88)-180 lbs | 90)-27.4 | |
| Medical Risk Factor- | Blood Pressure Treatment | N/A | 0 |
| Blood pressure | (step 96)-No | | |
| | SBP (step 98)-145 | | |
| | DBP (step 100)-90 | | |
| Medical Risk Factor- | Diabetes Treatment (step | N/A | 2 |
| Fasting plasma | 102)-No | | |
| glucose/Diabetes | Fasting Glucose (step | | |
| | 104)-80 mg/DL | | |
| Medical Risk Factor- | Total Cholesterol (step | N/A | 1 |
| Total cholesterol | 106)-220 mg/DL | | |
| | Treatment for Cholesterol | | |
| | (step 108)-No | | |
| Lifestyle Risk Factor- | Current Smoker (step 130)- | N/A | 2 |
| Smoking | No | | |
| | Quit within last 12 months | | |
| | (step 132)-No | | |
| Lifestyle Risk Factor- | Minutes per week | N/A | 1 |
| Physical activity* | moderate activity (step | | |
| | 134)-45 | | |
| | Minutes per week vigorous | | |
| | activity (step 136)-30 | | |
| Lifestyle Risk Factor- | Fruits and vegetables | (step 154a) 1 | 1 |
| Healthy diet score | (step 138)-6 | component | |
| | Fish (step 140)-3 | (step 154b) 0 | |
| | Fiber Rich Whole Grains | components | |
| | (step 142)-2 | (step 154c) 0 | |
| | Sodium (step 144)- | components | |
| | 1000 | (step 154d) 1 | |
| | Sugar Beverages (step | component | |
| | 146)-0 | (step 154e) 1 | |
| | | component | |
| Total CVH score | | | 8 |

As additional medical research is conducted, the data in Tables 1 and/or 2 may be modified to reflect more up-to-date research. Similarly, additional criteria may be added based on new medical research to alter the risk factors that are used to determine the CVH score, which may also result in a change in the CVH score scale described herein. Corresponding modifications to the method and system 10 (or 310) based on such new research may be made as will be understood by those of ordinary skill in the art.

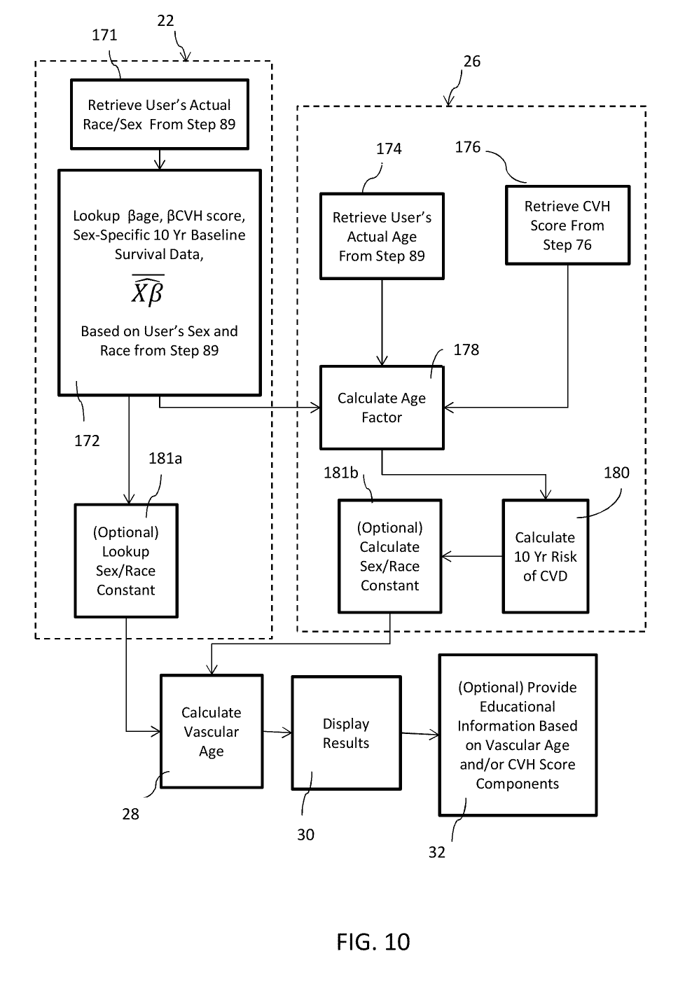

Referring to FIG. 10, the patient's personal data regarding race, sex, and age are retrieved at step 171. This data is used to lookup certain functional parameters or variables based on the patient's race and sex at step 172 that are used in calculating an age factor at step 178 and vascular age at step 28. Most preferably, the functional parameters for step 172 are from Tables 4A-4B, which was created based on data from four epidemiological studies, capturing four different ethnic groups: a) Whites—the Framingham Heart Study (FHS); b) Blacks—the Jackson Heart Study (JHS); c) Hispanics—the Northern Manhattan Study (NOMAS); and d) Chinese—the Multi-Ethnic Study of Atherosclerosis (MESA). For the Framingham Heart Study, second generation participants attending examination cycle 5 were used. Below is a description of each study.

FHS: All events were classified using hospital and other medical records, death certificates, and health status updates. A panel of physicians reviewed and adjudicated each event. MI was defined using ECG, enzymes, previous history and autopsy evidence, and was ascertained when there were at least two (2) of three (3) findings: 1) symptoms indicative of ischemia; 2) changes in biomarkers of myocardial necrosis; and 3) serial changes in the electrocardiograms. Stroke was defined as sudden or rapid onset of a focal neurologic deficit persisting for greater than 24 hours and is classified as hemorrhagic stroke, ischemic stroke, or athero-thrombotic brain infarction. Vascular death was defined as death from MI, CHD, and intermittent claudication.

JHS: At JHS, hospital and physician's office records were reviewed and adjudicated continuously. MI was defined by presence of cardiac pain, change in enzymes, and electrocardiographic findings. Hospitalized MI was defined using ICD-9 codes 402, 410-414, 427, 428, and 518.4. Ischemic stroke was defined based on ICD-9 code 435 and ICD-10 code G45. The criteria for classifying vascular death are based on any combination of 1) chest pain; 2) history of MI, CHD, or angina; 3) the absence of evidence of other probable cause of death; and/or 4) the use of ICD-9 codes (i.e., 250, 401, 402, 410-414, 427-429, 440, 518.4, 798, 799) or ICD-10 codes (E10-14, 110-11, 121-25, 146-51, 170, 197, J81, J96, R96, R98-99).

MESA: Periodic follow-ups of the cohort every 6-12 months were used to ascertain outcomes between examinations. Determination of events is performed using public files (death certificates), medical records from hospitalizations, autopsy reports, and interviews/questionnaires from participants regarding treatments and lifestyle changes, and in some cases, interviews/questionnaires from their physicians, friends and relatives. Events were reviewed by designated reviewers who determined the occurrence of the event and cause of death, and also discussed any discrepancies in diagnoses. If reviewers are unable to agree, the case was referred to the Morbidity and Mortality Committee. Hard cardiovascular disease is a composite variable defined by the first event of any myocardial infarction, resuscitated cardiac arrest, fatal/nonfatal stroke (not including TIA), or death from any CHD. Death from coronary heart disease included death from MI, CHD, stroke, and other cardiovascular disease death. Information on hard cardiovascular disease events was collected via participant phone interviews every 6-9 months and medical records.

NOMAS: Subjects were annually screened by phone to determine lifestyle changes, changes in vital status, detect stroke occurrence, document hospitalizations, and review status of risk factors, and treatment changes. People with a positive screening were scheduled for in-person assessment, including examination by a neurologist and also a chart review. Ongoing hospital records, including ICD-9 codes, and medical records were reviewed by specially trained research assistants (and study cardiologists and neurologists, when available) to verify events. MI was defined using criteria adapted from the Cardiac Arrhythmia Suppression Trial and the Lipid Research Clinics Coronary Primary Prevention Trial, requiring at least 2 of the 3 following criteria: (a) ischemic cardiac pain determined to be typical angina; (b) cardiac enzyme abnormalities defined as abnormal CPK-MB fraction or Troponin values; and (c) EKG abnormalities. Stroke was defined using the WHO criteria: “rapidly developing clinical signs of focal (at times global) disturbance of cerebral function, lasting more than 24 hours or leading to death with no apparent cause other than that of vascular origin.” Vascular death was defined using ICD-9 codes 390-459, medical records, death certificates and information from family.

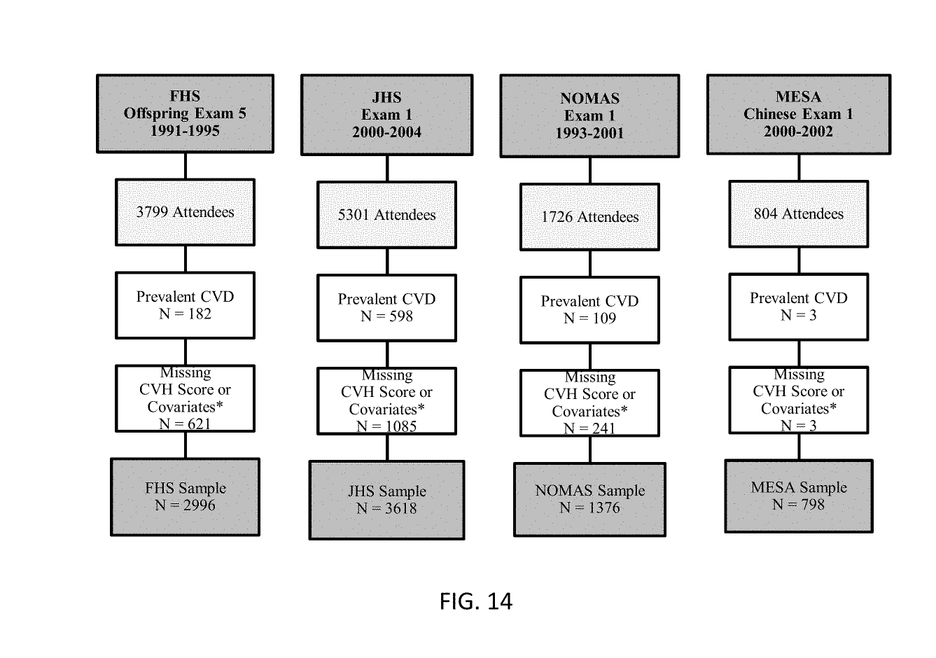

Follow-up time was truncated to a maximum of 13 years for FHS and NOMAS, in order to make the follow-up time ranges more homogeneous among the four cohorts. Tables 5A-5B show the descriptive characteristics for each cohort, and sample exclusions are presented in FIG. 14. A comparison of people included to those excluded (by cohort) is contained in Tables 6A-6B. MESA only excluded six subjects, so that cohort is not included in Tables 6A-6B.

TABLE 4A
Race/Sex-specific risk functions for a hard CVD event in 10-
years and derivation of vascular age

| | FHS (White) | | JHS (Black) | |
|---|---|---|---|---|
| | Women | Men | Women | Men |
| | N = 1619 | N = 1377 | N = 2421 | N = 1197 |
| CVD events, | 76 (4.7) | 138 (10.0) | 127 (5.2) | 67 (5.6) |
| N (%) | | | | |
| MI | 34 (44.7) | 74 (53.6) | 56 (44.1) | 36 (53.7) |
| Stroke | 35 (46.1) | 47 (34.1) | 64 (50.4) | 28 (41.8) |
| CVD death | 7 (9.2) | 17 (12.3) | 7 (5.5) | 3 (4.5) |
| Follow-up | 13.0 | 13.0 | 8.1 | 8.5 |
| time, yrs | (0.4, 13.0) | (0.1, 13.0) | (0.1, 11.1) | (0.1, 11.1) |
| Median | | | | |
| (Min, Max) | | | | |
| Function | | | | |
| parameters | | | | |
| \hat{β}_{age} ± SE | 0.070 ± | 0.062 ± | 0.069 ± | 0.055 ± |
| | 0.014 | 0.009 | 0.008 | 0.010 |
| \hat{β}_{CVH score} ± | −0.354 ± | −0.187 ± | −0.267 ± | −0.126 ± |
| SE | 0.053 | 0.044 | 0.051 | 0.061 |
| S₀(10) | 0.983 | 0.939 | 0.964 | 0.949 |
| [symbol] | 0.894 | 2.011 | 1.813 | 1.917 |
| Constant | 73.332 | 68.196 | 72.633 | 62.116 |
| term | | | | |
| C, (95% CI) | 0.777 | 0.720 | 0.788 | 0.731 |
| | (0.717, | (0.673, | (0.754, | (0.677, |
| | 0.837) | 0.768) | 0.821) | 0.786) |
| Calibration | 6.19 | 7.07 | 6.01 | 13.61 |
| X² | p = 0.72 | p = 0.63 | p = 0.74 | p = 0.14 |
| p-value | | | | |

TABLE 4B
Race/Sex-specific risk functions for a hard CVD event in 10-
years and derivation of vascular age

| | NOMAS (Hispanic) | | MESA (Chinese) | |
|---|---|---|---|---|
| | Women | Men | Women | Men |
| | N = 864 | N = 512 | N = 412 | N = 386 |
| CVD events, N | 149 (17.2) | 108 (21.1) | 20 (4.9) | 23 (6.0) |
| (%) | | | | |
| MI | 44 (29.5) | 37 (34.3) | 6 (30.0) | 11 (47.8) |
| Stroke | 67 (45.0) | 48 (44.4) | 7 (35.0) | 8 (34.8) |
| CVD death | 38 (25.5) | 23 (21.3) | 7 (35.0) | 4 (17.4) |
| Follow-up | 13.0 | 13.0 | 12.1 | 12.1 |
| time, yrs | (0.1, 13.0) | (0.1, 13.0) | (0.6, 13.4) | (0.2, 13.5) |
| Median (Min, | | | | |
| Max) | | | | |
| Function | | | | |
| parameters | | | | |
| \hat{β}_{age} ± SE | 0.085 ± | 0.072 ± | 0.114 ± | 0.065 ± |
| | 0.009 | 0.011 | 0.029 | 0.022 |
| \hat{β}_{CVH score} ± SE | −0.173 ± | −0.147 ± | −0.201 ± | −0.079 ± |
| | 0.045 | 0.049 | 0.114 | 0.122 |
| S₀(10) | 0.898 | 0.832 | 0.979 | 0.961 |
| [symbol] | 4.408 | 3.621 | 5.132 | 3.298 |
| Constant term | 76.280 | 74.8 | 66.179 | 57.569 |
| C, (95% CI) | 0.725 | 0.669 | 0.81 | 0.68 |
| | (0.686, 0.764) | (0.618, | (0.74, 0.89) | (0.59, 0.77) |
| | | 0.720) | | |
| Calibration X² | 6.25 | 2.05 | 11.68 | 18.54 |
| p-value | p = 0.71 | p = 0.99 | p = 0.23 | P = 0.03 |

As additional medical research is conducted, the data in Tables 4A-4B may be modified to reflect more up-to-date research. Although the system and method of the invention are currently configured for the four identified races, the information may still be useful for patients of other races. For example, patients of other Asian races may use the data for calculations based on the MESA study even though they are not Chinese. Similarly, the NOMAS data includes more Hispanics from certain countries than others, such as more Dominicans than Mexicans, and may not be as accurate for all Hispanics. The calculations will not be as accurate for other races as they are for the specific races in the studies upon which the calculations are based; however, the calculations may still provide a useful tool for understanding a patient's overall heart health.

TABLE 5A

| | FHS (White) | | JHS (Black) | |
|---|---|---|---|---|
| | Women | Men | Women | Men |
| N (%) | 1619 | 1377 | 2421 | 1197 |
| | (54.0) | (46.0) | (66.9) | (33.1) |
| Age, years | 54.6 ± 9.6 | 55.0 ± 9.8 | 53.9 ± 12.8 | 51.2 ± 12.6 |
| Body mass | 26.7 ± 5.5 | 28.2 ± 4.2 | 32.8 ± 7.7 | 29.8 ± 6.2 |
| index, kg/m² | | | | |
| Systolic blood | 123 ± 20 | 129 ± 17 | 125 ± 19 | 127 ± 17 |
| pressure, mmHg | | | | |
| Diastolic blood | 73 ± 10 | 77 ± 10 | 78 ± 10 | 82 ± 10 |
| pressure, mmHg | | | | |
| Hypertensive, N | 478 (29.5) | 506 (36.7) | 1423 (58.8) | 629 (52.5) |
| (%) | | | | |
| Antihypertensive | 265 (16.4) | 258 (18.7) | 1199 (49.6) | 417 (35.0) |
| medication, N | | | | |
| (%) | | | | |
| Total cholesterol, | 207 ± 38 | 202 ± 35 | 200 ± 39 | 197 ± 40 |
| mg/dL | | | | |
| HDL cholesterol, | 56 ± 15 | 43 ± 11 | 55 ± 14 | 46 ± 13 |
| mg/dL | | | | |
| Lipid-lowering | 85(5.3) | 105 (7.6) | 223 (9.2) | 84 (7.1) |
| medication, N | | | | |
| (%) | | | | |
| Fasting glucose, | 97.1 ± 24.5 | 103.9 ± 29.8 | 98.1 ± 31.9 | 98.8 ± 31.5 |
| mg/dL | | | | |
| Diabetes | 78 (4.8) | 114 (8.3) | 437 (18.1) | 170 (14.3) |
| mellitus, N (%) | | | | |
| Diabetes | 34 (2.1) | 52 (3.8) | 308 (13.5) | 108 (10.8) |
| medication, N | | | | |
| (%) | | | | |
| Ever smokers, N | 1000 (61.8) | 1038 (75.4) | 268 (11.1) | 260 (21.7) |
| (%) | | | | |
| Moderate | 1146 ± 841 | 1134 ± 848 | 42 ± 76 | 49 ± 84 |
| activity, | | | | |
| min/week* | | | | |
| Vigorous activity, | 208 ± 410 | 492 ± 697 | 20 ± 61 | 34 ± 83 |
| min/week* | | | | |
| Diet score | 1.7 ± 0.9 | 1.4 ± 0.9 | 1.3 ± 1.0 | 1.0 ± 1.0 |
| CVH score | 8.3 ± 2.2 | 7.6 ± 2.0 | 7.1 ± 2.0 | 7.2 ± 2.1 |
| CVH score, N | | | | |
| (%) | | | | |
| 0 | 0 (0.0) | 0 (0.0) | 0 (0.0) | 0 (0.0) |
| 1 | 2 (0.1) | 1 (0.1) | 1 (0.1) | 4 (0.3) |
| 2 | 7 (0.4) | 6 (0.4) | 11 (0.4) | 11 (0.9) |
| 3 | 26 (1.6) | 19 (1.4) | 58 (2.4) | 27 (2.3) |
| 4 | 54 (3.3) | 58 (4.2) | 157 (6.5) | 82 (6.8) |
| 5 | 90 (5.6) | 119 (8.6) | 329 (13.6) | 133 (11.1) |
| 6 | 175 (10.8) | 184 (13.4) | 408 (16.8) | 170 (14.2) |
| 7 | 225 (13.9) | 275 (20.0) | 437 (18.1) | 228 (19.1) |
| 8 | 257 (15.9) | 263 (19.1) | 392 (16.2) | 229 (19.1) |
| 9 | 276 (17.0) | 232 (16.8) | 293 (12.1) | 132 (11.0) |
| 10 | 231 (14.3) | 114 (8.3) | 206 (8.5) | 117 (9.8) |
| 11 | 165 (10.2) | 74 (5.4) | 82 (3.4) | 51 (4.3) |
| 12 | 92 (5.7) | 30 (2.2) | 42 (1.7) | 12 (1.0) |
| 13 | 18 (1.1) | 2 (0.1) | 5 (0.2) | 1 (0.1) |
| 14 | 1 (0.1) | 0 (0.0) | 0 (0.0) | 0 (0.0) |

Values represent mean ± standard deviation unless otherwise noted.
*Activity min/week in FHS are inflated, as activity was collected as hours/day and converted to min/week for this analysis. For this reason, FHS activity was treated differently in the calculation of the CVH score. For FHS only, poor health defined as <median of min/week moderate/vigorous intensity, intermediate health defined as median-top quartile of min/week moderate/vigorous intensity, and ideal health defined as top quartile of min/week moderate/vigorous intensity. This definition is quantitatively similar to the AHA physical activity metric. Median = 840 min/week, 75th percentile = 1260 min/week.

TABLE 5B

| | NOMAS (Hispanic) | | MESA (Chinese) | |
|---|---|---|---|---|
| | Women | Men | Women | Men |
| N (%) | 864 (62.8) | 512 (37.2) | 412 (51.6) | 386 (48.4) |
| Age, years | 65.9 ± 9.3 | 65.0 ± 8.5 | 62.3 ± 10.4 | 62.4 ± 10.3 |
| Body mass | 29.0 ± 5.2 | 27.3 ± 4.3 | 23.9 ± 3.4 | 24.1 ± 3.1 |
| index, kg/m² | | | | |
| Systolic blood | 143 ± 21 | 143 ± 22 | 125 ± 24 | 124 ± 19 |
| pressure, mmHg | | | | |
| Diastolic blood | 84 ± 10 | 86 ± 11 | 69 ± 11 | 75 ± 9 |
| pressure, mmHg | | | | |
| Hypertensive, N | 630 (72.9) | 353 (68.9) | 163 (39.6) | 135 (34.9) |
| (%) | | | | |
| Antihypertensive | 419 (48.5) | 180 (35.2) | 120 (29.1) | 109 (28.2) |
| medication, N | | | | |
| (%) | | | | |
| Total | 209 ± 40 | 193 ± 39 | 195 ± 32 | 190 ± 31 |
| cholesterol, | | | | |
| mg/dL | | | | |
| HDL cholesterol, | 47 ± 13 | 39 ± 12 | 53 ± 13 | 46 ± 11 |
| mg/dL | | | | |
| Lipid-lowering | 132 (15.3) | 47 (9.2) | 66 (16.0) | 50 (12.9) |
| medication, N | | | | |
| (%) | | | | |
| Fasting glucose, | 107.9 ± 52.7 | 108.9 ± 52.7 | 95.9 ± 23.9 | 102.0 ± 31.8 |
| mg/dL | | | | |
| Diabetes | 187 (21.6) | 113 (22.1) | 50 (12.1) | 53 (13.7) |
| mellitus, N (%) | | | | |
| Diabetes | 141 (16.3) | 83 (16.2) | 38 (9.2) | 38 (9.8) |
| medication, N | | | | |
| (%) | | | | |
| Ever smokers, N | 332 (38.4) | 344 (67.2) | 16 (3.9) | 181 (46.9) |
| (%) | | | | |
| Moderate | 93 ± 176 | 126 ± 235 | 245 ± 339 | 269 ± 351 |
| activity, | | | | |
| min/week* | | | | |
| Vigorous | 13 ± 82 | 19 ± 103 | 10 ± 52 | 33 ± 105 |
| activity, | | | | |
| min/week* | | | | |
| Diet score | 1.4 ± 1.0 | 1.0 ± 0.9 | 2.0 ± 0.7 | 1.7 ± 0.8 |
| CVH score | 6.9 ± 1.9 | 7.2 ± 1.9 | 9.8 ± 1.8 | 9.6 ± 1.8 |
| CVH score, N | | | | |
| (%) | | | | |
| 0 | 0 (0.0) | 0 (0.0) | 0 (0.0) | 0 (0.0) |
| 1 | 1 (0.1) | 2 (0.4) | 0 (0.0) | 0 (0.0) |
| 2 | 8 (0.9) | 2 (0.4) | 0 (0.0) | 0 (0.0) |
| 3 | 18 (2.1) | 11 (2.1) | 0 (0.0) | 0 (0.0) |
| 4 | 59 (6.8) | 26 (5.1) | 1 (0.3) | 1 (0.3) |
| 5 | 115 (13.3) | 51 (10.0) | 4 (1.0) | 2 (0.5) |
| 6 | 174 (20.1) | 77 (15.0) | 15 (3.7) | 14 (3.6) |
| 7 | 175 (20.3) | 112 (21.9) | 24 (5.8) | 29 (7.5) |
| 8 | 151 (17.5) | 113 (22.1) | 42 (10.2) | 69 (17.9) |
| 9 | 97 (11.2) | 61 (11.9) | 82 (19.9) | 62 (16.1) |
| 10 | 43 (5.0) | 35 (6.8) | 92 (22.3) | 77 (19.9) |
| 11 | 18 (2.1) | 17 (3.3) | 78 (18.9) | 72 (18.6) |
| 12 | 5 (0.6) | 5 (1.0) | 52 (12.6) | 42 (10.9) |
| 13 | 0 (0.0) | 0 (0.0) | 22 (5.3) | 18 (4.7) |
| 14 | 0 (0.0) | 0 (0.0) | 0 (0.0) | 0 (0.0) |

Values represent mean ± standard deviation unless otherwise noted.

TABLE 6A-1
Descriptive Characteristics of Included vs. Excluded
Subjects by Cohort-FHS (White)

| | Included | Excluded | | |
|---|---|---|---|---|
| | N = 2996 | N = 803 | | |
| | Mean ± std | Mean ± std | | |
| | or n (%) | or n (%) | N† | p-value |
| Female sex | 1619 (54.0) | 388 (48.3) | 803 | 0.004 |
| Age, years | 54.7 ± 9.7 | 58.2 ± 10.4 | 803 | <0.001 |
| BMI, kg/m² | 27.4 ± 5.0 | 27.7 ± 4.8 | 786 | 0.06 |
| SBP, mmHg | 126 ± 19 | 128 ± 19 | 802 | <0.001 |
| DBP, mmHg | 75 ± 10 | 75 ± 10 | 802 | 0.90 |
| Hypertensive | 984 (32.8) | 323 (40.3) | 802 | <0.001 |
| Antihypertensive | 523 (17.5) | 208 (26.4) | 789 | <0.001 |
| RX | | | | |
| Total | 205 ± 37 | 205 ± 38 | 774 | 0.62 |
| cholesterol, | | | | |
| mg/dL | | | | |
| HDL cholesterol, | 50 ± 15 | 48 ± 16 | 772 | <0.001 |
| mg/dL | | | | |
| Lipid-lowering | 190 (6.3) | 105 (13.1) | 801 | <0.001 |
| RX | | | | |
| Fasting glucose, | 100.2 ± 27.3 | 106.1 ± 37.6 | 772 | <0.001 |
| mg/dL | | | | |
| Diabetes | 192 (6.4) | 93 (12.0) | 773 | <0.001 |
| mellitus | | | | |
| Diabetes RX | 86 (2.9) | 54 (6.7) | 801 | <0.001 |
| Ever smokers | 2038 (68.0) | 595 (74.4) | 800 | <0.001 |
| Moderate | 1140 ± 844 | 1070 ± 820 | 686 | 0.05 |
| activity, | | | | |
| min/week | | | | |
| Vigorous | 339 ± 578 | 292 ± 596 | 685 | 0.06 |
| activity, | | | | |
| min/week | | | | |
| Diet score | 1.6 ± 0.9 | 1.6 ± 0.9 | 233 | 0.95 |
| CVH score | 8.0 ± 2.2 | 6.7 ± 2.0 | 125 | <0.001 |

N† = number of excluded subjects with available data

TABLE 6A-2
Descriptive Characteristics of Included vs. Excluded
Subjects by Cohort-JHS (Black)

| | Included | Excluded | | |
|---|---|---|---|---|
| | N = 3618 | N = 1683 | | |
| | Mean ± std | Mean ± std | | |
| | or n (%) | or n (%) | N† | p-value |
| Female sex | 2421 (66.9) | 946 (56.2) | 1683 | <0.001 |
| Age, years | 53.0 ± 12.8 | 60.5 ± 11.4 | 1683 | <0.001 |
| BMI, kg/m² | 31.8 ± 7.3 | 31.7 ± 7.0 | 1677 | 0.76 |
| SBP, mmHg | 126 ± 18 | 130 ± 19 | 1674 | <0.001 |
| DBP, mmHg | 79 ± 10 | 78 ± 11 | 1674 | <0.001 |
| Hypertensive | 2052 (56.7) | 1260 (75.3) | 1674 | <0.001 |
| Antihypertensive | 1616 (44.8) | 1096 (66.3) | 1652 | <0.001 |
| RX | | | | |
| Total | 199 ± 40 | 198 ± 41 | 1374 | 0.31 |
| cholesterol, | | | | |
| mg/dL | | | | |
| HDL cholesterol, | 52 ± 14 | 51 ± 15 | 1311 | 0.12 |
| mg/dL | | | | |
| Lipid-lowering | 307 (8.5) | 322 (19.5) | 1652 | <0.001 |
| RX | | | | |
| Fasting glucose, | 98.3 ± 31.8 | 109.8 ± 47.9 | 1640 | <0.001 |
| mg/dL | | | | |
| Diabetes | 607 (16.9) | 545 (33.2) | 1561 | <0.001 |
| mellitus | | | | |
| Diabetes RX | 416 (12.7) | 422 (27.0) | 1672 | <0.001 |
| Ever smokers | 528 (14.6) | 1188 (71.1) | 1678 | <0.001 |
| Moderate | 44 ± 79 | 43 ± 78 | 1678 | 0.63 |
| activity, | | | | |
| min/week | | | | |
| Vigorous | 25 ± 69 | 16 ± 49 | 1683 | <0.001 |
| activity, | | | | |
| min/week | | | | |
| Diet score | 1.2 ± 0.9 | 1.3 ± 1.0 | 1506 | <0.001 |
| CVH score | 7.2 ± 2.1 | 6.1 ± 2.0 | 355 | <0.001 |

N† = number of excluded subjects with available data

TABLE 6B
Descriptive Characteristics of Included vs. Excluded Subjects
by Cohort-NOMAS (Hispanic)

| | Included | Excluded | | |
|---|---|---|---|---|
| | N = 1376 | N = 350 | | |
| | Mean ± std | Mean ± std | | |
| | or n (%) | or n (%) | N† | p-value |
| Female sex | 864 (62.8) | 217 (62.0) | 350 | 0.78 |
| Age, years | 65.5 ± 9.0 | 69.3 ± 10.4 | 350 | <0.001 |
| BMI, kg/m² | 28.4 ± 5.0 | 28.3 ± 5.3 | 341 | 0.66 |
| SBP, mmHg | 143 ± 21 | 146 ± 21 | 344 | 0.06 |
| DBP, mmHg | 84 ± 11 | 84 ± 11 | 344 | 0.49 |
| Hypertensive | 983 (71.4) | 281 (80.3) | 350 | <0.001 |
| Antihypertensive | 599 (43.5) | 190 (54.4) | 349 | <0.001 |
| RX | | | | |
| Total | 203 ± 41 | 199 ± 43 | 289 | 0.14 |
| cholesterol, | | | | |
| mg/dL | | | | |
| HDL cholesterol, | 44 ± 13 | 43 ± 13 | 288 | 0.44 |
| mg/dL | | | | |
| Lipid-lowering | 179 (13.0) | 70 (20.1) | 349 | <0.001 |
| RX | | | | |
| Fasting glucose, | 108.3 ± 52.7 | 109.8 ± 54.2 | 269 | 0.67 |
| mg/dL | | | | |
| Diabetes | 300 (21.8) | 79 (22.6) | 350 | 0.76 |
| mellitus | | | | |
| Diabetes RX | 224 (16.3) | 61 (17.5) | 349 | 0.6 |
| Ever smokers | 676 (49.1) | 174 (49.7) | 350 | 0.84 |
| Moderate | 105 ± 200 | 174 ± 211 | 222 | <0.001 |
| activity, | | | | |
| min/week | | | | |
| Vigorous | 15 ± 91 | 69 ± 272 | 99 | 0.05 |
| activity, | | | | |
| min/week | | | | |
| Diet score | 1.2 ± 1.0 | 1.3 ± 1.1 | 174 | 0.36 |
| CVH score | 7.0 ± 1.9 | 6.6 ± 2.1 | 85 | 0.08 |

N† = number of excluded subjects with available data

The patient's age is also retrieved from step 89 at step 174, and the patient's CVH score is retrieved from step 76 at step 176. This information, along with the functional parameters based on race and sex from step 172, are used to calculate an age factor [symbol] at step 178. Most preferably, the age factor is calculated as follows:

[symbol]

=\hat{β}_{age}·X_{age}+\hat{β}_{CVH score}·X_{CVH score}−

[symbol]

where X_{age} is the patient's actual age retrieved from step 89, and X_{CVH score} is the patient's actual CVH score as determined at step 75. The other variables are the functional parameters retrieved from the data in Tables 4A-4B, based on the patient's sex and race, at step 172.

The age factor from step 178 is then used to calculate a 10 year risk of CVD (defined as myocardial infarction, stroke, or vascular death), \hat{p}, at step 180. Most preferably, the 10 year risk of CVD is calculated as follows:

\hat{p}=1−S₀(10

[symbol]

where S₀(10) indicates the sex-specific 10-year baseline survival, which is retrieved from the data in Tables 4A-4B based on the patient's race and sex, at step 172 and [symbol] is the age factor calculated at step 178. Next, the sex/race constant term is either looked-up from Tables 4A-4B at step 181a or is calculated at step 181b. If calculated, the constant term is preferably calculated as follows:

$$constant term = \frac{{\_}^{} - ( {\hat{β}}_{CVH score} \cdot 12 )}{{\hat{β}}_{Age}}$$

where all variables are functional parameters based on race and sex retrieved from the data in Tables 4A-4B at step 172.

Next, the patient's vascular age is calculated at step 28, using the constant term from step 181a or 181b, the 10 year risk of CVD calculated at step 180, and data retrieved from Tables 4A-4B, based on the patient's sex and race, at step 172. Most preferably, the patient's vascular age is calculated as follows:

$$Vascular Age = ln [ {( \frac{ln ( 1 - \hat{p} )}{ln ( {S}_{0} ( 10 ) )} )}^{\frac{1}{{\hat{β}}_{Age}}} ] + constant term$$

The results of the vascular age calculation are displayed at step 30 as a rounded number in years, representing the cardiovascular health age of the patient, which may be less than, greater than, or the same as the patient's actual age. An individual's vascular age (or heart age) is defined as the age of a hypothetical person with the same predicted risk of an incident CVD event in 10 years but with an ideal CVH score of 12. Although CVH score may go as high as 14, there were few subjects in the examined cohorts with scores higher than 12, so 12 is preferably used as indicating an ideal score. Additionally, using 12 as an ideal score (instead of 14) allows for individuals with very high CVH scores (13 and 14) to have vascular ages lower than their actual chronological age, which aids in creating a more understandable and relatable vascular age estimate.

As an example of the method 10, a patient with the following characteristics and risk factors will have a vascular age of 50 years.

TABLE 7
Example Patient

| Step | Characteristic/Risk Factor | Patient Data |
|---|---|---|
| 80 | Race | White |
| 82 | Sex | Female |
| 84 | Actual Age | 60 |
| 86 | Height | 5-8 |
| 88 | Weight | 150 |
| 90 | Calculated BMI | 22.8 |
| 94 | BMI Score | 2 |
| 96 | Treatment for Hypertension | No |
| 98 | Systolic BP | 115 |
| 100 | Diastolic BP | 70 |
| 102 | Treatment for Diabetes | No |
| 104 | Fasting Glucose | 90 |
| 106 | Total Cholesterol | 180 |
| 108 | Lipid Lowering Medication | No |
| 110 | BP Score | 2 |
| 112 | Glucose/Diabetes Score | 2 |
| 114 | Cholesterol Score | 2 |
| 130 | Smoking Status | No |
| 132 | Quit in last 12 months | No |
| 134 | Minutes moderate intensity activity | 160 |
| 136 | Minutes vigorous intensity activity | 80 |
| 138 | Cups fruits/vegetables per day | 5 |
| 140 | Fish per week | 10 |
| 142 | Fiber rich whole grains per day | 5 |
| 144 | Sodium per day | 1200 |
| 146 | Sweetened beverages per week | 0 |
| 150 | Smoking Score | 2 |
| 152 | Activity Score | 2 |
| 154 | Diet/nutrition score | 2 |
| 74 | CVH score | 14 |
| 172 | \hat{β}_{age} | 0.070 |
| 172 | \hat{β}_{CVH score} | −0.354 |
| 172 | [symbol] | 0.894 |
| 172 | S₀(10) | 0.983 |
| 181a | Constant term | 73.332 |
| 28 | Vascular Age | 50 years |

A test version of a vascular age calculator (using an Excel spreadsheet) demonstrated at an American Heart Association EPI Lifestyle Conference in March 2017 used 14 as the CVH score ideal value for making the calculations. With that ideal value, a hypothetical patient with the same health risk factors, race, sex, and age as indicated in Table 7 would have a vascular age of 60, equal to the patient's actual age. This was determined to not accurately reflect the excellent health of such a hypothetical patient, making the test version of the calculator less helpful in conveying cardiovascular health to a patient. The calculation method, including the functional parameters in Tables 4A-4B, was modified to lower the CVH ideal to 12 to allow the calculated vascular age according to the invention to be lower than the patient's actual age. Similarly, a lower ideal CVH score alters the vascular age calculation for patients across the majority of the CVH score scale. For example, a 60 year-old white woman with a CVH score of 12 (considered a high score) would have a vascular age of 70 years with a 14 point-score based ideal, but would have a vascular age of 60 (equal to actual age) with a 12 point-score based ideal. A 60 year-old white female with a CVH score of 6 would have a vascular age of 100 years using a 14 point-score based ideal, but would have a vascular age of 90 years using a 12 point-score based ideal. The following are the variables used in these example calculations:

14 point scale: Bage=0.070; Bcvh_score=−0.354; XB=0.894; S0(10)=0.983; Constant term=83.429

12 point scale: Bage=0.070; Bcvh_score=−0.354; XB=0.894; S0(10)=0.983; Constant term=73.332

The change in the constant term used make the calculation more accurate and meaningful in conveying a picture of a patient's cardiovascular health.

System and method 10 also preferably include step 32, at which the patient is provided with information based on the calculated vascular age and/or CVH score. Such information may be educational (such as information on ways to reduce blood pressure or cholesterol, smoking cessation materials, recipes, meal planning, diet tips, and/or exercise instruction), inspirational (words of encouragement or success stories from other patients who have improved their CVH score and/or vascular age), and/or medical (such as information pertaining to prescription drugs that may aid in reducing blood pressure, glucose levels, or cholesterol or information regarding clinical trials that a patient may be interested in following or becoming a participant). Most preferably, the information provided at step 32 is customized for a particular patient, based on responses to various inputs from step 20, the patient's CVH score, and/or the patient's vascular age; however, general health related information may also be provided, or a combination of general and customized information may be provided.

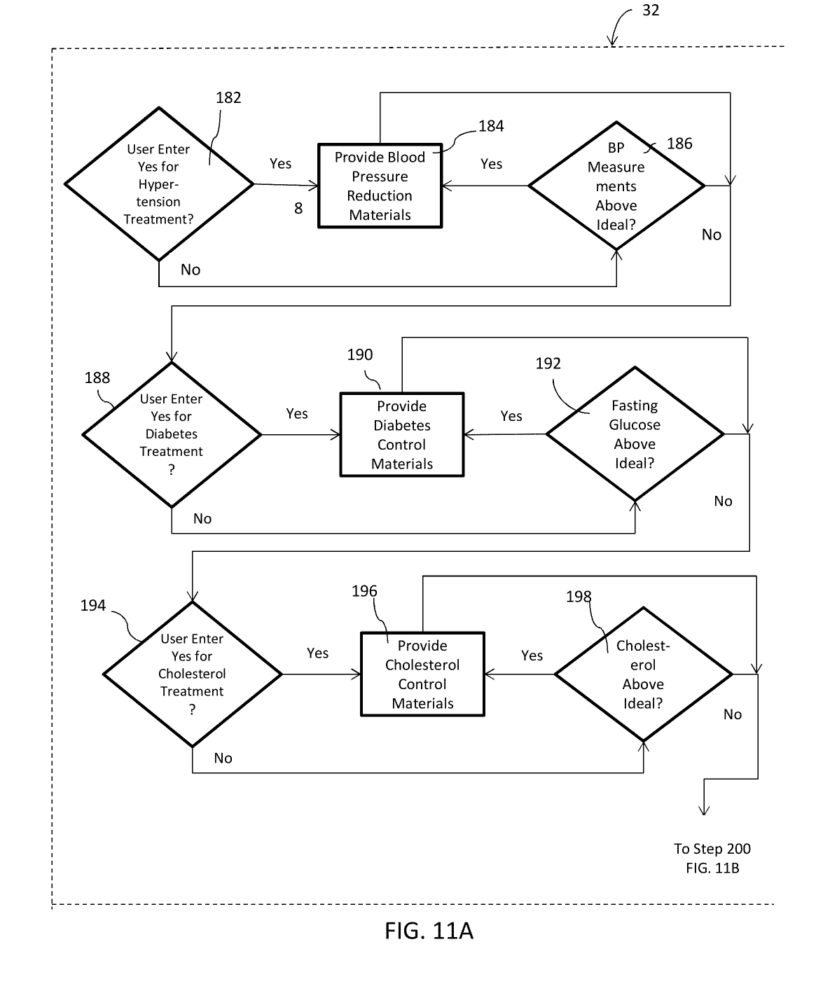

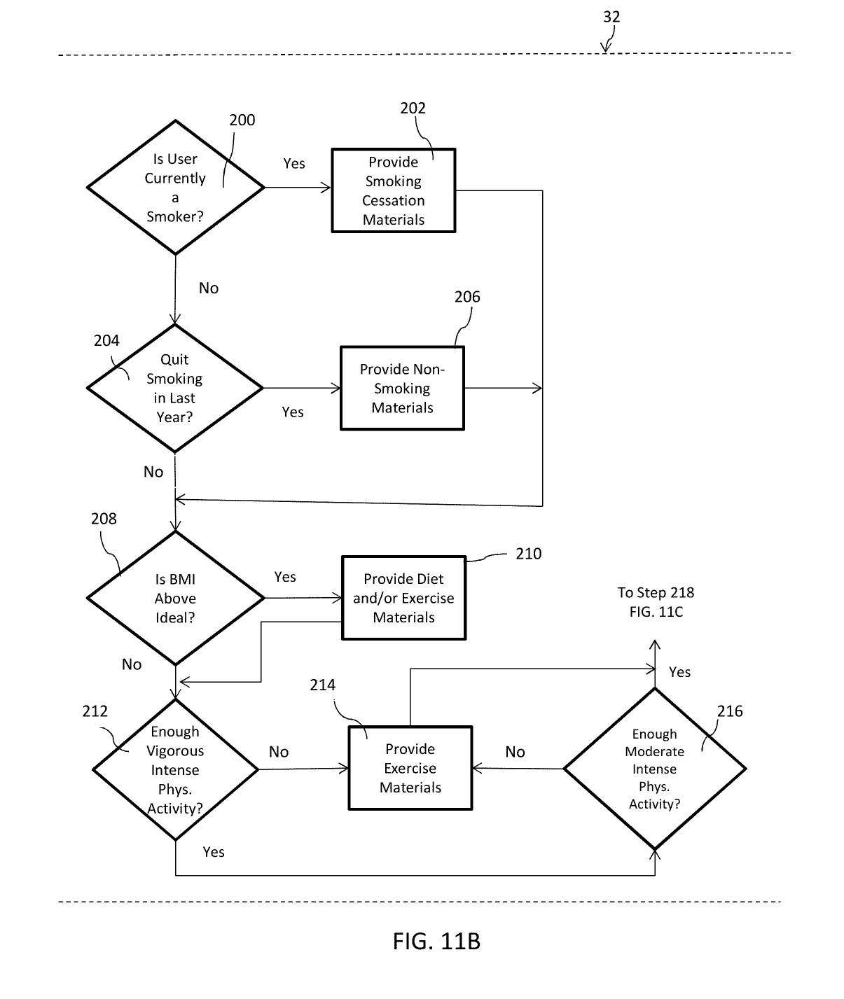

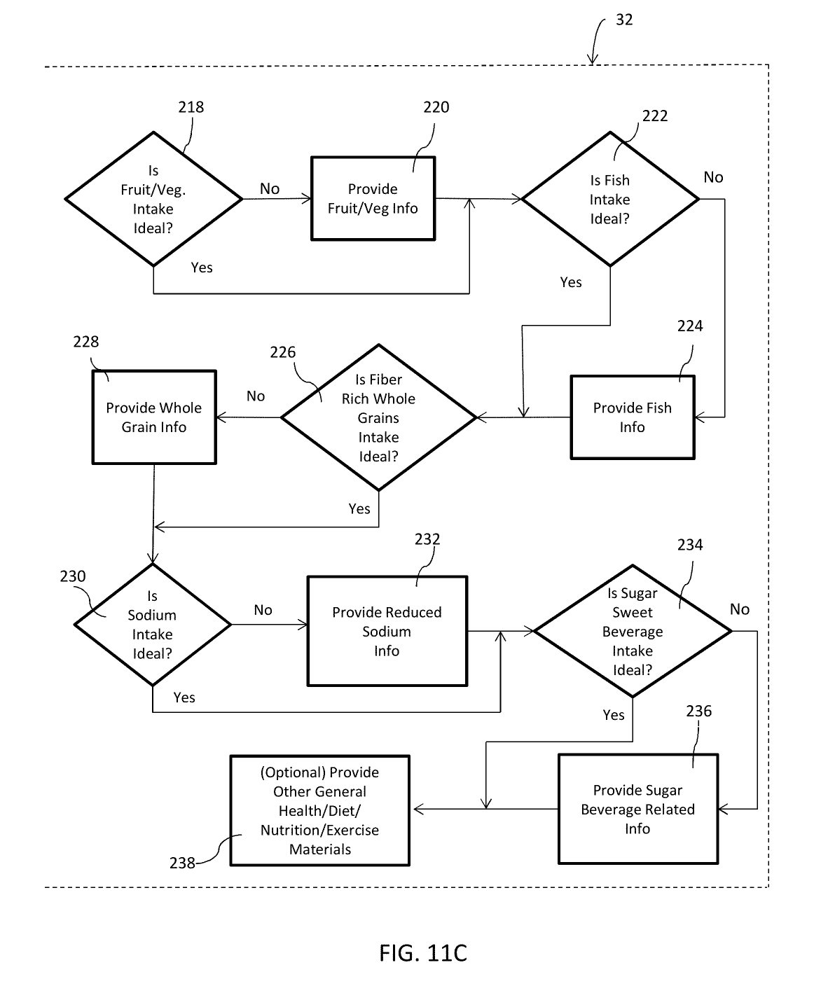

A preferred embodiment of step 32 for providing customized materials is show in FIGS. 11A-11C, wherein step 32 preferably comprises providing specific types of information based on the patient's data input. For example, at step 182, system 10 determines whether the user entered “yes” for hypertension treatment at step 96. If so, then the patient is optionally provided with blood pressure reduction materials at step 184, such as tips for reducing blood pressure. If not, then at step 186 system 10 determines whether the patient's blood pressure measurements (entered at steps 98 and 100) are above ideal. If so, then the patient is optionally provided with blood pressure reduction materials at step 184, such as tips for reducing blood pressure. If the patient is provided with blood pressure related materials at step 184, or the answers to both steps 182 and 186 are negative, then system 10 determines whether the user entered “yes” for diabetes treatment (entered at step 102) at step 188. If so, then the patient is optionally provided with diabetes related materials at step 190, such as recipes to help control blood glucose levels. If not, then at step 192 system 10 determines whether the patient's blood glucose measurement (entered at step 104) is above ideal. If so, then the patient is optionally provided with diabetes related materials at step 190, such as recipes to help control blood glucose levels.

If the patient is provided with diabetes related materials at step 190 or the answers to both steps 188 and 192 are negative, then system 10 determines whether the user entered “yes” for cholesterol treatment (entered at step 108) at step 194. If so, then the patient is optionally provided with cholesterol related materials at step 196, such as recipes to help reduce cholesterol levels. If not, then at step 198, system 10 determines whether the patient's total cholesterol measurement (entered at step 106) is above ideal. If so, then the patient is optionally provided with cholesterol related materials at step 196.

If the patient is provided with cholesterol related materials at step 196, or the answers to both steps 194 and 198 are negative, then system 10 determines whether the user entered “yes” for being a current smoker (entered at step 130) at step 200. If so, then the patient is optionally provided with smoking cessation related materials at step 202, such as tips on how to stop smoking and information regarding the dangers of smoking. If not, then at step 204 system 10 determines whether the patient stopped smoking in the last twelve months at step 204 (entered at step 132). If so, then the patient is optionally provided with smoking related materials at step 206, such as success stories of health improvement in persons who have stopped smoking or other support materials to help prevent the patient from returning to smoking.

If the patient is provided with smoking related materials at step 202 or 206, or the answers to both steps 200 and 204 are negative, then system 10 determines whether the patient's BMI (calculated at step 90) is above ideal at step 208. If so, then the patient is optionally provided with weight reduction or diet or exercise related materials at step 210, such as low calorie recipes or an exercise plan. If not, or if the patient is provided with materials at step 210, then at step 212, system 10 determines whether the patient's weekly vigorous activity level (entered at step 136) is ideal. If not, then the patient is optionally provided with exercise related materials at step 214, such as an exercise plan or success stories of health improvement in persons who have adopted exercise routines. If yes, then at step 216, system 10 determines whether the patient's weekly moderate activity level is ideal. If not, then the patient is optionally provided with exercise related materials at step 214. If yes, or if the patient is provided with materials at step 214, then system 10 determines at step 218 whether the patient's fruit and vegetable intake (entered at step 138) is ideal. If not, then the patient is optionally provided with fruit and vegetable related materials at step 220, such tips for incorporating more vegetables into the patient's diet or recipes, and proceeds to step 222. If yes, then system 10 determines at step 222 whether the patient's fish intake (entered at step 140) is ideal. If not, then the patient is optionally provided with fish related materials at step 224, such as tips for incorporating more fish into the patient's diet or information regarding supplements, and proceeds to step 226. If yes, then then system 10 determines at step 226 whether the patient's fiber rich whole grain intake (entered at step 142) is ideal. If not, then the patient is optionally provided with whole grain related materials at step 228, such as tips for incorporating more fiber rich whole grains into the patient's diet or recipes, and proceeds to step 230. If yes, then system 10 determines at step 230 whether the patient's sodium intake (entered at step 144) is ideal. If not, then the patient is optionally provided with sodium related materials at step 232, such as reduced sodium recipes, and proceeds to step 234. If yes, then system 10 determines at step 234 whether the patient's sugar sweet beverage intake (entered at step 146) is ideal. If not, then the patient is optionally provided with sugar sweetened beverage related materials at step 236, such as alternatives to sugar sweetened drinks, and proceeds to step 238. If yes, then system 10 optionally provides at step 238 other general health or diet or nutrition or exercise related materials to the patient.

Materials or information provided in step 32 may include any type of health and lifestyle related materials, including nutrition information, exercise information, recipes, diet information, grocery lists, meal planning information, smoking cessation information, tips for reducing sodium intake, cholesterol control information, diabetes control information, blood pressure control information, weight management information, weight loss information, stress reduction information, and information regarding prescription medications. Materials or information provided in step 32 may be provided to the patient in any form, such as displayed on a screen, printouts, sent by email, download, uploaded to a file sharing site, text, or other electronic means, or may be sent by postal mail. They may also optionally be sent to a healthcare provider to give to and/or review with the patient at the patient's next appointment with the provider. The comparison to ideal steps shown in FIGS. 11A-11B need not be separate steps, but may be incorporated into the comparison steps used in determining CVH score. For example, step 152 may be used to determine whether a patient should receive exercise related materials at step 214, instead of using separate steps 212 and 216.

According to another preferred embodiment, step 32 comprises using the CVH sub-score for one or more risk factors to determine whether a patient should receive materials. If any given sub-score is below a predetermined threshold, then the patient will receive materials related to that particular risk factor. Most preferably, the predetermined threshold is a “2,” the highest possible sub-score, but a sub-score below 1 may also be used to trigger providing materials related to that risk factor. For example, if the CVH sub-score for cholesterol at step 120 is less than ideal (less than 2), then the patient should receive cholesterol related materials at step 196, without having to repeat the comparison steps 194 and 198 that were used to determine the sub-score at step 120.

According to another preferred embodiment, step 32 comprises providing generalized health information when the patient's calculated vascular age is more than a predetermined amount over the patient's actual age. For example, if the vascular age is more than 5 years over the patient's actual age or is 5% or 10% higher than the patients' actual age, then the patient will be provided with health information to aid the patient in improving overall health and lowering his/her vascular age for future calculations. Other predetermined parameters may also be used to trigger step 32 based on calculated vascular age. According to another preferred embodiment, step 32 comprises providing generalized health information when the patient's CVH score, or optional My Life Check score, is below a predetermined threshold. For example, if the CVH score is 9 or below or the My Life Check score is 7 or below, then the patient will be provided with health information to aid the patient in improving overall health and lowering his/her CVH score or My Life Check score for future calculations.

Referring to FIGS. 12-13, another preferred embodiment of a method and system 310 is shown. In this preferred embodiment, a patient may create a login profile and the system may store the patient's data and calculations for future reference. At step 312, the user is prompted to enter whether the patient is a returning user. If not, the user is sent to step 20 and proceeds through the previously described steps, including optionally creating a profile to save the patient's data for future use, to determine the patient's vascular age. If yes, the user is prompted to enter his/her login information. The user is then prompted at step 316 to enter whether there have been any changes to the patient's age, height, and/or weight since the last time data for the patient was entered. Any change in age may also be calculated by system 310 based on the current date and the patient's age entered the last time data for the patient was entered, or based on the current date and the patient's date of birth (if included as part of step 84). If there have not been any changes, then at step 320 the patient's previous BMI data is retrieved. If there have been changes, then at step 318 steps 84, 86, 88, 89, 62, and 64 are repeated. At step 322, the user is prompted to indicate whether there have been any changes to the patient's medical risk factors. If there have not been any changes, then the previous medical data is retrieved at step 326. If there have been changes, then steps 44, 66, and 68 are repeated. At step 328, the user is prompted to indicate whether there have been any changes to the patient's lifestyle risk factors. If there have not been any changes, then the previous lifestyle data is retrieved at step 332. If there have been changes, then steps 46, 70 and 72 are repeated.

With the retrieved previous data and new risk factor data, step 24 is repeated at step 334 to determine a new CVH score. The CVH score may optionally be converted to a My Life Check Score at step 336. If the data in Tables 4A-4B has been changed since the last vascular age calculation, step 22 is repeated at step 344 based on the new data. If not, the previously looked-up functional parameters may be retrieved if previously stored for this particular patient. Step 26 is repeated at step 346 using current age, new CVH score from step 334, and functional parameter data from step 344 to determine a new age factor and new 10 year risk of CVD. This data is then used at step 348 to calculate a new vascular age by repeating step 28 using the equations indicated above. The new vascular age is displayed or otherwise provided at step 354. Optionally, informational materials are provided at step 356 by repeating step 32 based on the new data and results.

Optionally, the previous (or one or more previous) CVH scores (or My Life Check scores) and/or vascular ages may be retrieved at steps 338 and 350 and compared to the new CVH score (and new My Life Check Score) and vascular age at steps 340 and 352. The new CVH score, and optional My Life Check Score, and/or results of a comparison to previous scores may optionally be displayed or otherwise provided to the user at step 342. Similarly, the results of a comparison of current vascular age to one or more previously determined vascular age results may also be displayed or otherwise provided at step 354. Any comparison to previous scores is preferably displayed or provided as a percentage of change, a line or bar graph showing current and previous results, or a single number indicating the changed amount (e.g. new vascular age is 5 years younger than previous vascular age or new CVH score is 3 points higher than previous score). Most preferably, if a patient creates a login profile, the patient's data is saved for future use and comparison to aid in the patient's understanding of how changes in risk factors impact CVH score and vascular age.

Each of steps 316, 322, and 328 may be grouped to prompt the user to indicate a change in more than one type of information. For example, step 322 could prompt the user to indicate yes or no to whether there have been changes to blood pressure, cholesterol, or glucose measurements or medications. If the user enters “yes,” then all of steps 44, 66, and 68 have to be repeated. According to another preferred embodiment, the user is separately prompted to indicate whether there is a change for each type of information in steps 316, 322, and 328. For example, a user would be separately prompted for change in height, change in weight, changes in blood pressure measurements and medication status, changes in cholesterol measurement and medication status, etc. Having separate prompts would reduce the number of steps that may have to be repeated. For example, if the user indicates no change in blood pressure measurement or medications, but that there have been changes in cholesterol measurement or medications, then steps 106, 108, 114, and 120 related to cholesterol would be repeated, but it would not be necessary to repeat steps 96, 98, 100, 110, or 116 related to blood pressure.

According to one preferred embodiment, a system for carrying out the preferred method of heart health assessment and communication according to the invention comprises:
one or more computers or terminals or other computing devices, with or without a server, accessible by a user/patient/healthcare provider, to allow data entry in response to prompts regarding personal information and risk factors; to provide or display a CVH score and/or vascular age to the user/patient/healthcare provide; optionally to receive or allow for printing or download of risk factor specific and/or generalized health information; and/or optionally to allow communication via the internet with one or more external sites (an external computer, terminal, or computing device, an external database, an externally hosted software site, file sharing site, and/or a healthcare provider computer, terminal, or computing device);
a software component at the user/patient/healthcare provider's computer, terminal, computing device, or server or remotely accessible from such (hosted at the software provider's or other third party's site) for managing, initiating, and carrying out the method steps of the personal data and risk factor data entry, calculations, and optional provision of information;
a database system or similar data storage capability for storing data used to determine CVH score and the race/sex data used to make other calculations, including the data contained in Tables 1, 2, and 4A-4B, the database system accessible by the software component or integrated into the software component;
if the software component is hosted externally from the user/patient/healthcare provider's device, a computerized server connected to a network to host the software, preferably containing the database system and optional database systems, and to allow communication with and between one or more user computers, terminals, or other computing devices; one or more users/patients, including by email; and optionally one or more external sites (an external database, file sharing site, or a remote healthcare provider or patient computer or terminal) via the internet;
an optional database system or similar data storage capability for storing files containing historic or previously entered personal information and risk factor data, previous calculations of CVH score, optionally My Life Check Score, and vascular age, the database system accessible by the software component; and
an optional database system or similar data storage capability for storing risk factor related information and/or generalized health information, the database system accessible by the software component to send, display, or otherwise provide such information to the user/patient/healthcare provider, preferably through the user/patient/healthcare provider's computer, terminal, computing device, or server.

Computer, terminals, and computing devices preferably have a processor and memory storage capability to allow execution of the software component to carry out the steps of the methods according to the invention. Methods and systems 10 and 310 may also be incorporated into a number of medical related devices, such as devices to measure a medical parameter of the patient. For example, an automated blood pressure measuring machine (similar to those found at pharmacies) or a weight scale or a home blood glucose testing device, may incorporate various components or embodiments of a method and system 10 according to the invention. When incorporated into such devices, certain patient inputs may be skipped. For example, if part of an automated blood pressure measuring device, the patient's blood pressure would be actually measured and those measurements used in place of prompting the user to enter those measurements at steps 98 and 100. Various embodiments of a method according to the invention may also be carried out on a computer, tablet, smart phone, stand-alone kiosk, or similar device having a user interface to allow input of a patient's data, a processor for making calculations, and the ability to store data (such as Tables 4A and 4B and, optionally, a patient's particular data for a given date).

The basic operation and calculations made in methods and systems 10 and 310 may also be incorporated into a pre-existing medical database or patient health record system, which already includes basic information regarding the patient's race, sex, age, height, weight, and medical history, which may be periodically updated with new physical exams or data entry. Systems 10 and 310 may be modified to retrieve such pre-existing information directly from such medical database or patient health record system without requiring separate data entry, as will be understood by those of ordinary skill in the art.

One or more components or steps described with one preferred embodiment of a system or method according to the invention may be used with another embodiment of the system or method, even if not specifically described in connection with the embodiment. References to servers, computers, terminals, and other computing devices are intended to include all such devices that permit the data entry, communication, storage, and access to data and information, and/or that are capable of carrying out steps defined in a software program. Steps described herein as a prompt to a user may alternatively be a tab, drop-down box, or other option to be selected by the user or recipient. Unless a specific format is indicated (e.g. display on a screen), references herein to displaying or providing results or information include any manner of display or providing the results or information, such as displayed on a screen, printouts, postal mail, courier, sent by email, download, uploaded to a file sharing site, text, or other electronic means. Any combination of optional stages or steps may be used in connection with the preferred methods and system of the invention. Those of ordinary skill in the art will also appreciate upon reading this specification, that modifications and alterations to the methodology and system for calculation of a vascular age may be made within the scope of the invention and it is intended that the scope of the invention disclosed herein be limited only by the broadest interpretation of the appended claims to which the inventors are legally entitled.

What is claimed is:

1. A method for assessing a patient's heart health, the method comprising:
obtaining information regarding the patient's race, sex, and age and regarding a plurality of personal risk factors for the patient;
determining a CVH score for the patient;
retrieving functional parameters based on the patient's race and sex;
determining a vascular age for the patient based on the patient's CVH score and the retrieved functional parameters;
providing the vascular age to the patient; and
providing health information to the patient based on the information regarding one or more of the plurality of personal risk factors, CVH score, vascular age, or a combination thereof.

2. The method of claim 1 wherein the determining the CVH score step comprises:
comparing the information regarding the plurality of personal risk factors to a set of ideal values for each risk factor;
assigning a point value for each of the plurality of personal risk factors based on the comparison; and
adding the point values together to calculate the patient's CVH score.

3. The method of claim 2 wherein the personal risk factors comprise at least one risk factor from each of the following categories: physical risk factors, medical risk factors, and lifestyle risk factors; and
wherein the ideal values for each risk factor are those listed in Tables 1 and 2.

4. The method of claim 3 wherein the physical risk factors comprise height and weight or BMI;
wherein the medical risk factors comprises one or more of the following: whether the patient is on medication for treatment of high blood pressure, lipids, or diabetes, systolic blood pressure measurement, diastolic blood pressure measurement, total cholesterol measurement, and fasting blood glucose measurement; and
wherein the lifestyle risk factors comprise one or more of the following: whether the patient is a current smoker, whether the patient quit smoking in the last twelve months, amount of physical activity per predetermined time period, amount of fruits and vegetables consumed per predetermined time period, amount of fish consumed per predetermined time period, amount of fiber rich whole grains consumed per predetermined time period, amount of sodium consumed per predetermined time period, and amount of sugar sweet beverages consumed per predetermined time period.

5. The method of claim 1 wherein the functional parameters based on sex and race are listed in Tables 4A-4B.

6. The method of claim 5 wherein the determining a vascular age step comprises:
calculating an age factor for the patient using the equation: [symbol]
=\tilde{β}_{age}·X_{age}+\hat{β}_{CVH score}·X_{CVH score}− [symbol]
calculating a 10 year risk of CVD for the patient using the equation:
\hat{p}=1−S₀(10 [symbol] ; and
calculating the patient's vascular age using the equation:
$$Vascular Age = ln [ {( \frac{ln ( 1 - \hat{p} )}{ln ( {S}_{0} ( 10 ) )} )}^{\frac{1}{{\hat{β}}_{Age}}} ] + constant term$$
wherein X_{age} is the patient's actual age, X_{CVH score} is the determined CVH score for the patient, and all other variables are functional parameters based on the patient's race and sex.

7. The method of claim 6 wherein the information regarding personal risk factors comprises three or more of: height, weight, measured blood pressure, measured cholesterol, and measured fasting blood glucose; and
wherein the obtaining step comprises data entry, retrieving information from a patient health record system, or a combination thereof.

8. The method of claim 6 further comprising:
identifying one or more personal risk factors where the assigned point value is below a predetermined threshold; and
wherein the providing health information step comprises providing health information specific to the identified personal risk factor.

9. The method of claim 8 wherein the health information comprises one or more of nutrition information, exercise information, recipes, diet information, grocery lists, meal planning information, smoking cessation information, tips for reducing sodium intake, cholesterol control information, diabetes control information, blood pressure control information, weight management information, weight loss information, stress reduction information and information regarding prescription medications.

10. The method of claim 6 further comprising determining whether the patient's calculated vascular age is over the patient's actual age by a predetermined amount or whether the patient's CVH score is under a predetermined threshold or both; and
wherein the providing health information step is carried out when the patient's calculated vascular age is over the patient's actual age by a predetermined amount or whether the patient's CVH score is under a predetermined threshold or both.

11. A method for assessing a patient's heart health, the method comprising:
obtaining information regarding the patient's race, sex, and age and regarding a plurality of personal risk factors for the patient;
determining a CVH score for the patient;
retrieving functional parameters based on the patient's race and sex as listed in Tables 4A-4B;
determining a vascular age for the patient based on the patient's CVH score and the retrieved functional parameters; and
providing the vascular age to the patient.

12. The method of claim 11 wherein the information regarding the plurality of risk factors comprises BMI, cholesterol information, diabetes information, blood pressure information, smoking information, activity level information, and diet information;
wherein the determining the CVH score step comprises:
comparing the risk factor information to an ideal value or set of ideal values for each risk factor;
assigning a point value for each of the personal risk factors based on the comparison; and
adding the point values together to calculate the patient's CVH score on a scale of 0 to 14.

13. The method of claim 12 wherein the determining a vascular age step comprises:
calculating an age factor for the patient using the equation: [symbol]
=\hat{β}_{age}·X_{age}+\tilde{β}_{CVH score}·X_{CVH score}− [symbol]
calculating a 10 year risk of CVD for the patient using the equation:
\hat{p}=1−S₀(10 [symbol] ; and
calculating the patient's vascular age using the equation:
$$Vascular Age = ln [ {( \frac{ln ( 1 - \hat{p} )}{ln ( {S}_{0} ( 10 ) )} )}^{\frac{1}{{\hat{β}}_{Age}}} ] + constant term$$
wherein X_{age} is the patient's actual age, X_{CVH score} is the determined CVH score for the patient, and all other variables are functional parameters based on the patient's race and sex.

14. The method of claim 13 further comprising:
identifying one or more personal risk factors where the assigned point value is below a predetermined threshold; and
providing health information specific to any personal risk factor identified.

15. The method of claim 14 wherein the health information comprises one or more of nutrition information, exercise information, recipes, diet information, grocery lists, meal planning information, smoking cessation information, tips for reducing sodium intake, cholesterol control information, diabetes control information, blood pressure control information, weight management information, weight loss information, stress reduction information and information regarding prescription medications.

16. The method of claim 13 further comprising determining whether the patient's calculated vascular age is over the patient's actual age by a predetermined amount or whether the patient's CVH score is under a predetermined threshold or both; and
providing health information if the patient's calculated vascular age is over the patient's actual age by the predetermined amount or if the patient's CVH score is under the predetermined threshold or both.

17. A system for assessing a patient's heart health, the system comprising:
a computer, terminal, or other computing device accessible by a user or patient;
a software component on or accessible from the computer, terminal or other computing device configured to (1) obtain information regarding the patient's race, sex, and age; (2) obtain information regarding a plurality of personal risk factors for the patient; (3) determine a CVH score for the patient; (4) retrieve functional parameters based on the patient's race and sex; (5) determine a vascular age for the patient based on the patient's CVH score and the retrieved functional parameters; (6) provide the vascular age to the patient; and (7) optionally provide health information to the user or patient;
one or more database systems or data storage systems for storing ideal values for each risk factor, functional parameters based on race and sex as listed in Tables 4A-4B, optionally previously obtained information regarding the personal risk factors for the patient, optionally previously determined CVH score, optionally previously determined vascular age, optionally health information, or a combination thereof.

18. The system according to claim 17 wherein the software component is configured to determine a CVH score by comparing the risk factor information to an ideal value or set of ideal values for each risk factor;
assigning a point value for each of the personal risk factors based on the comparison; and
adding the point values together to calculate the patient's CVH score on a scale of 0 to 14; and
wherein the software component is configured to determine a vascular age by L calculating an age factor for the patient using the equation: [symbol]
=\hat{β}_{age}·X_{age}+\hat{β}_{CVH score}·X_{CVH score}− [symbol]
calculating a 10 year risk of CVD for the patient using the equation:
\hat{p}=1−S₀(10 [symbol] ; and
calculating the patient's vascular age using the equation:
$$Vascular Age = ln [ {( \frac{ln ( 1 - \hat{p} )}{ln ( {S}_{0} ( 10 ) )} )}^{\frac{1}{{\hat{β}}_{Age}}} ] + constant term$$
wherein X_{age} is the patient's actual age, X_{CVH score} is the determined CVH score for the patient, and all other variables are functional parameters based on the patient's race and sex.

19. The system of claim 18 wherein the computer, terminal, or other computing device or the software component is integrated into a device to measure a medical parameter of the patient or integrated into a patient health record system.
